(19) United States
(12) Patent Application Publication
(10) Pub. No.: US 2009/0286001 A1
(43) Pub. Date: Nov. 19, 2009

(54) ACTIVE-ENERGY RADIATION-POLYMERIZABLE SUBSTANCE, ACTIVE-ENERGY RADIATION-CURABLE LIQUID COMPOSITION, ACTIVE-ENERGY RADIATION-CURABLE INK, INK JET RECORDING METHOD, INK CARTRIDGE, RECORDING UNIT, AND INK JET RECORDING APPARATUS

(75) Inventors: Tsuyoshi Kanke, Yokohama-shi (JP); Yutaka Kurabayashi, Higashimurayama-shi (JP); Kenji Shinjo, Yokohama-shi (JP); Hiromitsu Kishi, Kawasaki-shi (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/064,132

(22) PCT Filed: Sep. 28, 2006
(86) PCT No.: PCT/JP2006/319922

§ 371 Date: Feb. 19, 2008

(30) Foreign Application Priority Data
Sep. 30, 2005 (JP) 2005-287799

(51) Int. Cl. C08J 7/18; C07D 207/24; C07D 207/12; C08F 2/46; B41J 2/175; B41J 2/01

(52) U.S. Cl. 427/487; 548/521; 548/520; 522/167; 522/84; 347/86; 347/102

(57) ABSTRACT

An active energy radiation polymerizable substance is disclosed which is represented by the following general formula (I): wherein Z is a dihydric to hexahydric polyol residue, and A, B and D are groups represented by the following formulas (II) to (IV), respectively: (II), (III), and (IV).

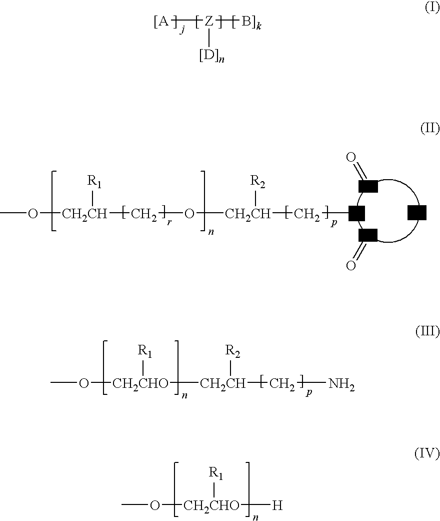

### TECHNICAL FIELD

This invention relates to a novel active-energy radiation-polymerizable substance, an active-energy radiation-curable liquid composition, an active-energy radiation-curable ink, an ink-jet recording method, an ink cartridge, a recording unit and an ink jet recording apparatus.

### BACKGROUND ART

Techniques using aqueous coating materials or inks have conventionally been known in methods in which a resin composition in an ink is cured by irradiation with light including an active-energy radiation to form resin cured films to form images. In regard to an active-energy radiation-polymerizable substance used in this case, which is used in a material constitution of the aqueous coating material or ink, such techniques as shown below are known in the art. For example, techniques are known in which a nonaqueous active-energy radiation-polymerizable substance is used to be formulated into an emulsion in an aqueous medium, and in which an ultraviolet curable resin and a polymerization initiator are made aqueous.

Techniques are also known in which a liquid composition, or an ink, containing such an active-energy radiation-polymerizable substance is applied to an ink jet recording method. In recent years, the active-energy radiation-curable liquid composition and the active-energy radiation-curable ink are applied to, e.g., graphic art, signs, displays, label recording, package recording, electronic circuit boards, and fabrication of display panels.

In the case where the active-energy radiation-curable ink is used in such an ink jet recording method, it may be contemplated to use a nonaqueous or aqueous resin composition. The non-aqueous resin composition is known to be roughly classified into two types of inks as typical ones. One of the two types is known to be what is called an oil-based ink composed of an organic solvent such as toluene or methyl ethyl ketone and a pigment dispersed therein. Another type is known to be what is called a 100%-curable ink not using any organic solvent and containing a monomer, an oligomer and a pigment dispersion (i.e., non-solvent ink). Where, however, the above oil-based ink is used, sufficient consideration must be taken for environment because the organic solvent volatilizes in air. The 100%-curable ink creates a difference in roughness between recorded areas and non-recorded areas, i.e., unevenness of images as a whole on recording mediums, and hence it is difficult to attain a feeling of gloss on images, and under the existing conditions, it is difficult to put the 100%-curable ink forward into use where high image quality is required.

However, such techniques of curing by an active-energy radiation are in fact expected as curing techniques that are energy-saving and reduce environmental pollution and environmental burden. Further, the utilization of the techniques of curing by an active-energy radiation in ink jet recording is considered useful not only in the recording of images but also in the pre-treatment to provide recording base materials with recording suitability and in the post-treatment to coat them with materials for protecting and processing recording mediums on which images have been formed. In addition, by applying the technique for an aqueous ink which is generally used in ink jet recording to the active-energy radiation-curable technique, it is possible to alleviate the unevenness of images that is the problem the above 100%-curable ink has, and hence it is advantageous from the viewpoint of making image quality higher. Under such circumstances, it is sought to develop a hydrophilic resin, a polyfunctional monomer and a monofunctional monomer which are applicable also to active-energy radiation-curable aqueous inks for ink jet recording.

In order to apply the materials to ink jet recording methods, it is required for the materials to have low viscosity and good flow properties which are adaptable to high-density nozzles. For example, it is sought to increase the content of a polymerizable substance to some extent in an ink. It is also sought to shorten the time for drying after the ink has been applied to a recording medium. It is further sought to provide a hydrophilic resin, a polyfunctional monomer and a monofunctional monomer which are superior in physical properties of cured ink films (ink layers, i.e. recorded areas) and have good compatibility with coloring materials. Of these, in regard to the monomer, especially in respect, of the polyfunctional monomer from the viewpoints of polymerization rate and physical properties of films formed after polymerization, it is sought to develop high-performance materials.

As an example of active-energy radiation-curable monomers, a hydrophilic polymerizable substance having an acidic group and a (meth)acryloyl group or a vinyl group is known as a compound having one polymerizable functional group in one molecule. Such a compound may include, e.g., an ester of succinic anhydride with 2-hydroxyethyl (meth)acrylate, an ester of orthophthalic anhydride with 2-hydroxyethyl (meth)acrylate, and vinylnaphthalene sulfonic acids.

As another example, a polymerizable substance provided with hydrophilicity by a polyethylene oxide chain is known as a compound soluble in water, having two or more polymerizable functional groups in one molecule and produced in, an industrial scale. Such a compound may include, e.g., (meth)acrylates of, polyhydric alcohols, such as diethylene glycol (meth)acrylate, and tetraethylene glycol di(meth)acrylate.

Japanese Patent Application Laid-open No. H08-165441 discloses a polyfunctional hydrophilic polymerizable substance. The compound disclosed therein is a compound obtained by using a method in which the number of hydroxyl groups in its molecule is increased to provide hydrophilicity.

Japanese Patent Applications Laid-open No. 2000-117960 and No. 2002-187981 disclose (meth)acrylates of hydrophilic polyoxides derived from polyalcohols. Compounds disclosed in these documents can achieve to a certain extent the polymerizability by an active-energy radiation and the physical properties of cured products, and also the viscosity shown when the compound is formulated into an aqueous solution satisfies the level required for ink jet recording inks.

Further, Japanese Patent Application Laid-open No. 2003-165927 discloses an energy radiation-curable composition for powder coating materials which has, in addition to a (meth)acrylate compound containing a spiro ring, a compound containing an ethylenically unsaturated group.

However, since the above compound having one polymerizable functional group in one molecule has only one polymerizable functional group in one molecule, its polymerization rate is low and its cured product have a very low degree of cross-linking. Accordingly, that compound is difficult to use as a chief material for hydrophilic active-energy radiation-curable materials.

The above compound soluble in water, having two or more polymerizable functional groups in one molecule and produced in an industrial scale, has been found, according to studies made by the present inventors, to have such problems as stated below. That is, such a compound may be low in hydrophilicity if it has a short ethylene oxide chain. If on the other hand it has a long ethylene oxide chain, it can have hydrophilicity, but when polymerized or cured, the cured product may have insufficient physical properties as a solid in respect of performance such as hardness and adherence required for coating materials and inks.

The compound disclosed in Japanese Patent Application Laid-open No. H08-165441 has been found, according to studies made by the present inventors, to have such problems as stated below. That is, such a compound may certainly have superior polymerizability by an active-energy radiation and its cured product have superior physical properties, but a problem is raised in that its aqueous solution has somewhat higher viscosity than the level required for ink jet recording inks.

The compounds disclosed in Japanese Patent Applications Laid-open No. 2000-117960 and No. 2002-187981 have been found, according to studies made by the present inventors, to have such problems as stated below. Where a dye capable of dissolving in an aqueous medium in virtue of an anionic group or a pigment dispersion in which a pigment has been dispersed in an aqueous medium in virtue of an anionic group is used as a coloring material for an ink containing a hydrophilic polymerizable substance having a (meth)acrylate group, a problem as stated below may arise. That is, the pH of the ink is lowered to an acid region as an acrylic acid is formed due to hydrolysis of the (meth)acrylate group. The dye or the pigment dispersion, having been stably present in the ink where the pH of the ink is in an alkaline to neutral region, is precipitated or agglomerated. Thus, there may be a problem in view of storage stability of inks.

The compound disclosed in Japanese Patent Application Laid-open No. 2003-165927 is used as a composition for powder coating materials, and is unclear as to whether the compound is hydrophilic or water-soluble. Further, the compound is unclear also as to whether sufficient curability is achieved, because it is described that a monofunctional maleimide compound or the like is preferred.

In an ink jet recording method in which the ink is ejected by the action of thermal energy, polymerizable substance in ink brings about heat polymerization due to thermal energy, so that polymers insoluble in water are formed, in nozzles. Thus, there may be a problem in view of ejection stability of inks.

### DISCLOSURE OF THE INVENTION

A first object of the present invention is to provide an active-energy radiation-polymerizable substance which is rapidly polymerizable by an active-energy radiation, ensures a high degree of cross-linking of cured products formed, and is not substantially hydrolyzed even when formulated into an aqueous liquid composition or an ink.

A second object of the present invention is to provide an active-energy radiation-curable liquid composition which is rapidly polymerizable by an active-energy radiation, ensure a high degree of cross-linking of cured products formed, and has superior adherence to recording mediums.

A third object of the present invention is to provide an active-energy radiation-curable ink which achieves the second object, and also has a viscosity satisfying the level of viscosity required in ink jet recording methods, and is superior in storage stability.

A fourth object of the present invention is to provide an ink jet recording method using an active-energy radiation-curable ink which achieves the second and third objects, and also does not bring about any heat polymerization due to thermal energy to have reduced influence on the ejection of the ink, and is superior in ejection stability.

A fifth object of the present invention is to provide an ink cartridge including a ink storage portion in which the above active-energy radiation-curable ink is stored, and a recording unit and an ink jet recording apparatus which use the above active-energy radiation-curable ink.

The above objects are achieved by the invention described below. That is, as a first embodiment according to the first object of the present invention, an active-energy radiation-polymerizable substance is provided which is represented by the following general formula (I).

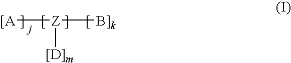

In the general formula (I), Z is a residue of a dihydric to hexahydric polyol, j is 1 to 6, k is 0 to 2, and m is 0 to 2.

In the general formula (I), A is a group represented by the following general formula (II).

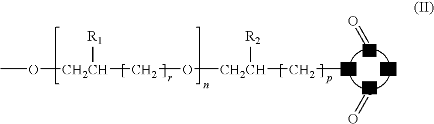

In the general formula (II), n is 0 to 5; p is 0 to 1; R₁ and R₂ are each independently a hydrogen atom, a methyl group or a hydroxyl group; r is 0 to 1; and X is a divalent group constituted of 2 to 5 carbon atoms in which at least one of the carbon atoms adjoining to the carbonyl carbons has a carbon-carbon double bond.

In the general formula (I), B is a group represented by the following general formula (III).

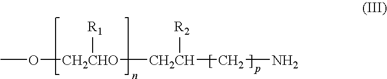

In the general formula (III), n is 0 to 5; p is 0 to 1; and R₁ and R₂ are each independently a hydrogen atom, a methyl group or a hydroxyl group.

In the general formula (I), D is a group represented by the following general formula (IV).

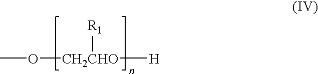

In the general formula (IV), n is 0 to 5; and R₁ is a hydrogen atom, a methyl group or a hydroxyl group.

In the present invention, —X— in the general formula (II) may preferably be a group represented by the following chemical formula (1) or (2).

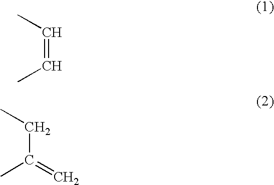

The present invention also provides as a second embodiment an active-energy radiation-curable liquid composition characterized by containing at least the active-energy radiation-polymerizable substance represented by the general formula (I).

The present invention provides as a third embodiment an active-energy radiation-curable ink characterized by containing at least the active-energy radiation-polymerizable substance represented by the general formula (I), and a coloring material. This ink is preferable as an ink jet recording ink (hereinafter referred to simply as “ink” in some cases).

The present invention provides as a fourth embodiment an ink jet recording method having the step of ejecting an ink to apply the ink to a recording medium and the step of irradiating the recording medium to which the ink has been app lied, with an active-energy radiation to cure the ink, wherein the ink is the ink of the present invention, described above.

The present invention provides as a fifth embodiment the following ink cartridge, recording unit and ink jet recording apparatus. More specifically, the present invention provides an ink cartridge having an ink storage portion in which the ink of the present invention as described above is stored. The present invention also provides a recording unit characterized by having an ink storage portion in which the ink of the present invention as described above is stored, and a recording head for ejecting the ink. The present invention further provides an ink jet recording apparatus characterized by having a means for applying the ink of the present invention as described above to a recording medium and a means for irradiating the recording medium to which the ink has been applied, with an active-energy radiation to cure the ink.

According to the first embodiment of the present invention, an active-energy radiation-polymerizable substance can be provided which is rapidly polymerizable by an active-energy radiation, ensure a high degree of cross-linking of cured products formed, and bring about substantially no hydrolysis.

According to the second embodiment of the present invention, an active-energy radiation-curable liquid composition can be provided which is rapidly polymerizable by an, active-energy radiation, insure a high degree of cross-linking of cured products formed, and is superior in adherence to recording mediums.

According to the third embodiment of the present invention, an active-energy radiation-curable ink can be provided which has a viscosity satisfying the level of viscosity required in ink jet-recording methods and is superior in storage stability.

According to the fourth embodiment of the present invention, an ink jet recording method can be provided which uses an active-energy radiation-curable ink which does not bring about any heat polymerization due to thermal energy to have reduced influence on the ejection of the ink, and is superior in ejection stability.

According to the fifth embodiment of the present invention, an ink cartridge, a recording unit and an ink jet recording apparatus can be provided which uses the active-energy radiation-curable ink.

### BRIEF DESCRIPTION OF THE DRAWING

FIG. 1 is a schematic front view of a preferred ink jet recording apparatus used in the present invention.

### BEST MODE FOR CARRYING OUT THE INVENTION

The present invention is described below in greater detail by means of preferred embodiments.

Taking the above objects into account, the present inventors have made various studies. As a result, they have discovered the active-energy radiation-polymerizable substance represented by the general formula (I) (hereinafter referred to simply as “polymerizable substance” in some cases) that is radically polymerizable by an active-energy radiation. Then, they have prepared an active-energy radiation-curable ink (hereinafter referred to simply as “ink” in some cases) containing the polymerizable substance represented by the general formula (I), to evaluate this active-energy radiation-curable ink. As a result, they have discovered that the active-energy radiation-curable ink having such a constitution has superior curing performance involved with the degree of cross-linking and adherence of cured products obtained, and has a viscosity satisfying the level of low viscosity required in ink jet recording methods and is superior in storage stability and ejection stability. Thus, they have arrived at the present invention.

It is unclear why good results are obtained not only on curing performance but also on storage stability and ejection stability of the ink. The present inventors presume the reason as stated below.

In regard to the storage stability of the ink, it is presume as follows. The active-energy radiation-polymerizable substance of the present invention, represented by the general formula (I), can not easily be affected by hydrolysis in an aqueous solution, compared with conventional polymerizable substances having an acrylate structure, and maleic acid and so forth are formed in a very small quantity. Hence, the pH of the ink is kept from being lowered to an acid region. Accordingly, the dye capable of dissolving in an aqueous medium in virtue of an anionic group or the pigment dispersion in which a pigment has been dispersed in an aqueous medium in virtue of an anionic group can maintain the stably dissolved or dispersed state. As a result, the ink can be presumed to have superior storage stability.

In regard to the ejection stability of the ink, it is presumed as follows. The active-energy radiation-polymerizable substance of the present invention, represented by the general formula (I), has a higher resistance to heat polymerization, or the acid produced by hydrolysis is in a very-smaller quantity, when compared with conventional polymerizable substances having an acrylate structure. For any of these reasons, the active-energy radiation-polymerizable substance itself is kept from heat-polymerization. As a result, the ink is presumed to have superior ejection stability.

It will be explained below how the active-energy radiation-polymerizable substance operates and is effective, in aqueous ink jet recording that is a primary example to which this polymerizable substance is applicable. In the present invention, ultraviolet rays or electron rays may be used as the active-energy radiation. In the following description, an ultraviolet-curable ink capable of curing through radical polymerization caused by ultraviolet rays, which is particularly preferably usable in the present invention, is taken up as an example. It is a matter of course that in the present invention, the active-energy radiation used for curing are by no means limited to the ultraviolet rays.

The chief objects for which the active-energy radiation-curable ink of the present invention is used as an ink used in the ink jet recording method are as follows: for example, (1) to improve the drying performance of the ink to adapt the ink to an improvement in recording speed; (2) to provide the polymerizable substance with a function as a dispersant of the coloring material, to form images having superior scratch resistance on various recording mediums; (3) to reduce light-scattering of pigment particles to form transparent ink layers; (4) to enlarge the range of color reproduction of process colors and to make the ink high in optical density and superior in chroma and brightness; and (5) to protect coloring Materials from active light, gas components in air, water, and so forth.

Especially in a recording medium on the one hand having ink absorptivity on the one hand, but on the other hand being difficult to improve in pigment hues and scratch resistance, as in the case of plain paper, the ink of the present invention can exhibit a remarkable effect of remedying such problems. Of course, such an effect is not limited only to plain paper, and the same effect as in plain paper is obtainable also on recording mediums having smaller ink absorptivity, as exemplified by offset paper, business form paper and corrugated fiberboard. Moreover, the ink of the present invention makes it possible to record on non-absorptive recording mediums.

Curing by an active-energy radiation is one of forced-drying methods, and can be said to be a method in which the state of an ink applied to a recording medium such as paper is frozen before the ink permeates completely into the recording medium, i.e., during the time that the ink is forming a free surface. In the ink of the present invention, it comes that the permeation and evaporation of an aqueous medium such as water proceeds gradually from ink layers having come into solids. However, apparent drying takes place quickly as stated above, and hence the fixing time in the sense that the recording medium can be transported, or mounted can be handled as having come short. However, as long as the aqueous medium is used, it is inevitable that true drying comes slower than in the case of inks using organic solvents. Accordingly, in using the ink of the present invention, a final forced heat dryer may be furnished, depending on uses.

It is important purely as a problem of radical-reaction rate how the curing proceeds in the polymerizable substance, as in the ink of the present invention, radically polymerized by the active-energy radiation in the state the water is present. According studies made by the present inventors, in the case of colorless inks containing no coloring material, it has not been observed that the polymerization reaction of the polymerizable substance in water is especially slower than the case of non-solvent systems. Of course, since the polymer formed contains water, the cured product has solid physical properties different from those in the case of non-solvent systems.

The active-energy radiation-polymerizable substance, active-energy radiation-curable liquid composition and active-energy radiation-curable ink of the present invention, having superior operations and effects as stated above, are described below.

—Active-Energy Radiation-Polymerizable Substance—

The active-energy radiation-polymerizable substance of the present invention may preferably be hydrophilic. In the present invention, what is referred to as “compound is hydrophilic” means that the compound is in any one of the following states. (1) The compound is soluble in an organic solvent miscible with water, and the organic solvent is water-soluble. (2) Even if the compound is not water-soluble, it has been so treated as to be emulsifiable with water. (3) The compound is water-soluble.

The active-energy radiation-polymerizable substance of the present invention is represented by the following general formula (I).

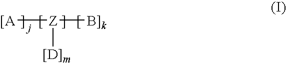

In the general formula (I), Z is a residue of a dihydric to hexahydric polyol, j is 1 to 6, k is 0 to 2, and m is 0 to 2.

In the general formula (I), [A] is a group represented by the following general formula (II).

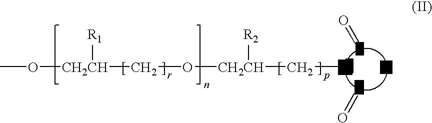

In the general formula (II), n is 0 to 5; p is 0 to 1; R₁ and R₂ are each independently a hydrogen atom, a methyl group or a hydroxyl group; r is 0 to 1; and X is a divalent group constituted of 2 to 5 carbon atoms in which at least one of the carbon atoms adjoining to the carbonyl carbon has a carbon-carbon double bond.

In the general formula (II), X may preferably be a group represented by the following chemical formula (1) or (2).

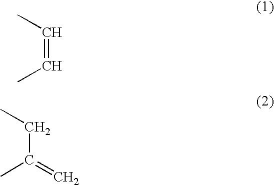

As an example of the group represented by the general formula (II), it may include a group represented by the following general formula (II′).

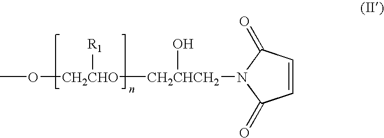

In the general formula (II′), n is 0 to 5, and R₁ is a hydrogen atom or a methyl group.

As another example of the group represented by the general formula (II), it may include a group represented by the following general formula (II″).

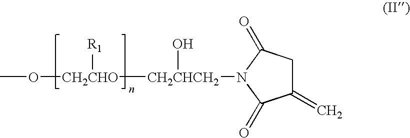

In the general formula (II″), n is 0 to 5, and R₁ is a hydrogen atom or a methyl group.

[B] in the general formula (I) is a group represented by the following general formula (III).

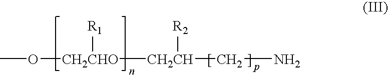

In the general formula (III), n′ is 0 to 5; p is 0 to 1; and R₁ and R₂ are each independently a hydrogen atom, a methyl group or a hydroxyl group.

As an example of the group represented by the general formula (III), it may include a group represented by the following general formula (III′).

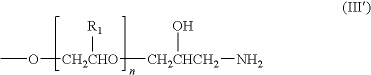

In the general formula (III′), n is 0 to 5, and R₁ is a hydrogen atom or a methyl group.

[D] in the general formula (I) is a group represented by the following general formula (IV).

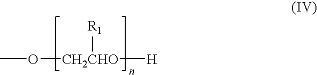

In the general formula (IV), n is 0 to 5; and R₁ is a hydrogen atom, a methyl group or a hydroxyl group.

The number of polymerizable functional groups of the active-energy radiation-polymerizable substance may preferably be 2 or more and 6 or less, more preferably 3 or more and 6 or less, and particularly preferably 3 or more and 4 or less. The larger the number of polymerizable functional groups in the polymerizable substance, the more improved the curing performance is. However, with an increase in the number of polymerizable functional groups, the ink has a higher viscosity, so that any active-energy radiation-curable ink may be not obtainable which is adaptable to high-density nozzles of a recording head and has good flow properties. If on the other hand the number of polymerizable functional groups in the polymerizable substance is small, the polymerization rate is so low that the cured product may have a very low degree of cross-linking.

It is particularly preferable that the active-energy radiation-polymerizable substance represented by the general formula (I) has both an ethylene oxide group and a propylene oxide group in its molecular structure. This is because, inasmuch as it has both an ethylene oxide group and a propylene oxide group, the viscosity can remarkably be made low when formulated into an aqueous solution, in virtue of the steric hindrance of the propylene oxide group, to afford especially superior ejection stability and storage stability.

The hydrophilicity of the active-energy radiation-polymerizable substance is provided by the ethylene oxide chain or propylene oxide chain and the hydroxyl group contained in the groups represented by the general formulas (II), (III) and (IV). The number (n) of the ethylene oxide chains or propylene oxide chains contained in the groups represented by the general formulas (II), (III) and (IV) may preferably be in the range of from 0 to 5, and more preferably in the range of from 1 to 3. The number of the ethylene oxide chains or propylene oxide chains may have distribution. If the ethylene oxide chains or propylene oxide chains in the active-energy radiation-polymerizable substance are short, the polymerizable substance has low hydrophilicity. If on the other hand the ethylene oxide chains or propylene oxide chains are long, the polymerizable substance can have hydrophilicity, but the cured product may have insufficient solid physical properties in respect of performance such as hardness or adherence.

The polyol residue represented by [Z] in the general formula (I) is a polyol from which one or more hydroxyl groups have been removed. Preferred polyols may specifically include, e.g., the following: Ethylene glycol, diethylene glycol, triethylene glycol and tetraethylene glycol; polyethylene glycols having an average molecular weight of 200 or more and 5,000 or less, such as polyethylene glycol (PEG) 200, PEG 300, PEG 400, PEG 600, PEG 1,000 and PEG 2,000; propylene glycol, dipropylene glycol, tripropylene glycol, and polypropylene glycol having an average molecular weight of 230 or more and 5,000 or less; 1,3-propanediol, 1,2-butanediol, 1,3-butanediol, 1,4-butanediol and 2,3-butanediol; 1,5-pentanediol, 1,4-pentanediol and 2,4-penanediol; 3-methyl-1,5-pentanediol and 2-methyl-2,4-penanediol; 1,5-hexanediol, 1,6-hexanediol, 2,5-hexanediol and glycerol; 1,2,4-butanetriol, 1,2,6-hexanetriol and 1,2,5-pentanetriol; thiodiglycol, trimethylolpropane, ditrimethylolpropane, trimethylolethane, ditrimethylolethane, neopentyl glycol, pentaerythritol, and condensates thereof. In the present invention, it is necessary that the number of residues of polyols is from 2 to 6 (i.e. [Z] is a residue of a dihydric to hexahydric polyol).

The ethylene oxide chains or propylene oxide chains which are the unit constituting the above polyethylene glycols or polypropylene glycols are obtained by polymerization reaction. Hence, the number of the ethylene oxide chains or propylene oxide chains has distribution, and the number and molecular weight of these units in the molecule are expressed as average values.

Other polyols may specifically include, e.g., the following: Polyvinyl alcohol; monosaccharides or deoxy sugars thereof, such as triose, tetrose (erythritol, threitol) and pentose (ribitol, arabinitol, xylitol); other monosaccharides or deoxy sugars thereof, such as hexose (allitol, allitritol, glucitol, mannitol, iditol, galactitol, inocitol), heptose, octose, nonose and decose; and aldonic acid or aldaric acid derivatives. Of these, it is particularly preferable to use glycerol, 1,2,4-butanetriol, 1,2,6-hexanetriol, 1,2,5-pentanetriol, trimethylolpropane, trimethylolethane, neopentyl glycol or pentaerythritol. Of course, in the present invention, examples are by, no means limited to these. In the present invention, it is particularly preferable that, in the general formula (I), the value of j+k+m is equal to the number of residues of the polyol, i.e., j+k+m=2 to 6.

The active-energy radiation-polymerizable substance represented by the general formula (I) may include, as particularly preferable examples, active-energy radiation-polymerizable substances having structures shown below. Of course, active-energy radiation-polymerizable substances usable in the present invention are by no means limited to these. These compounds are highly hydrophilic, are polymerizable and are high in polymerization, and have a low viscosity in themselves. At the same time, when formulated into an aqueous solution, they have a viscosity which is markedly lower than conventionally known compounds.

In the present invention, two or more kinds of active-energy radiation-polymerizable substances may be used in combination. For example, to explain it in relationship to Exemplified Compounds 2 and 3 given below, maleimide groups are exemplified as terminal groups having radical polymerizability. When designing the active-energy radiation-curable ink, the ink may be required to be designed taking various aspects into account. For example, there are problems of the viscosity of ink and the strength of cured films. For such problems, it is possible to balance by, e.g., using Exemplified Compounds 2 and 3 in the form of a mixture. In some cases, a monofunctional monomer corresponding to Exemplified Compound 3 in which one maleimide group has been introduced may be used in combination. Instead, a compound having quite different terminal groups, as exemplified by a monomer in which reactive terminal groups are imides derived from itaconic acid, may be used in combination. Thus, there are no particular limitations concerning the polyfunctional monomer or monofunctional monomer usable in combination in the present invention as long as satisfying what is defined in the present invention. The active-energy radiation-polymerizable substance may also be used in combination with a conventionally known hydrophilic monomer or water-dispersible monomer.

### Exemplified Compound 1

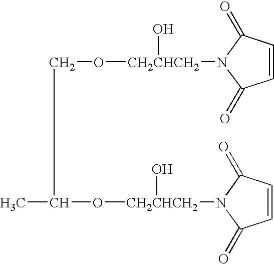

In the Exemplified Compound 1, Z of the general formula (I) corresponds to a propylene glycol residue (shown below).

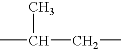

### Exemplified Compound 2

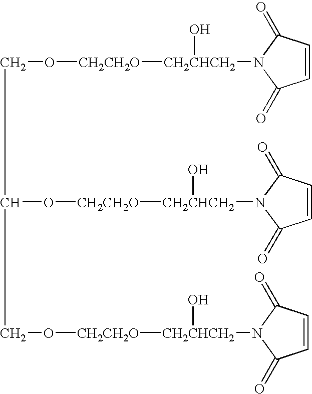

In the Exemplified Compound 2, Z of the general formula (I) corresponds to a glycerol residue (shown below).

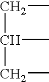

### Exemplified Compound 3

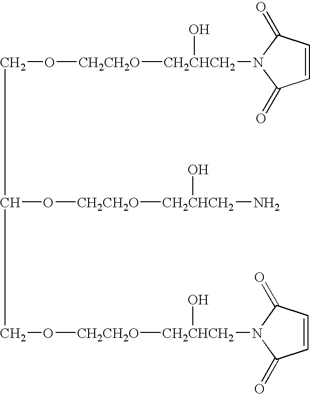

In the Exemplified Compound 3, Z of the general formula (I) corresponds to a glycerol residue (shown below).

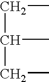

As the positions at which the maleimide groups are introduced, where the carbon atoms of the glycerol residue are defined as the 1st, 2nd and 3rd carbon atoms from the top, the terminals of the groups bonded to the 1st and 3rd carbon atoms are shown in Exemplified Compound 3, but the maleimide groups may be bonded to the terminals of the groups bonded to the 1st and 2nd (or 2nd and 3rd) carbon atoms. It is applied to all the following Exemplified Compounds without regard to the number of substituents that such substituent isomers fall under the category of this Exemplified Compound.

### Exemplified Compound 4

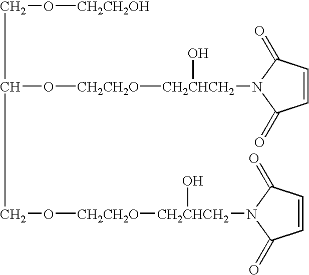

In the Exemplified Compound 4, Z of the general formula (I) corresponds to a glycerol residue (shown below).

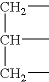

### Exemplified Compound 5

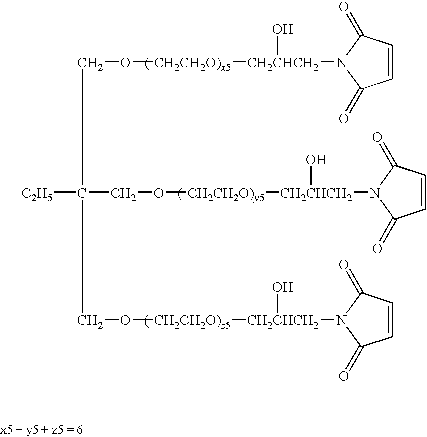

In the Exemplified Compound 5, Z of the general formula (I) corresponds to a trimethylolpropane residue (shown below).

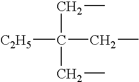

### Exemplified Compound 6

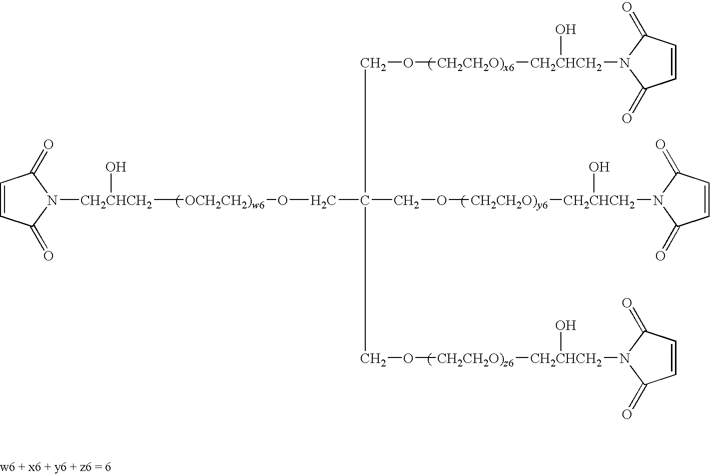

In the Exemplified Compound 6, Z of the general formula (I) corresponds to a pentaerythritol residue (shown below).

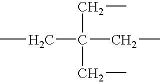

### Exemplified Compound 7

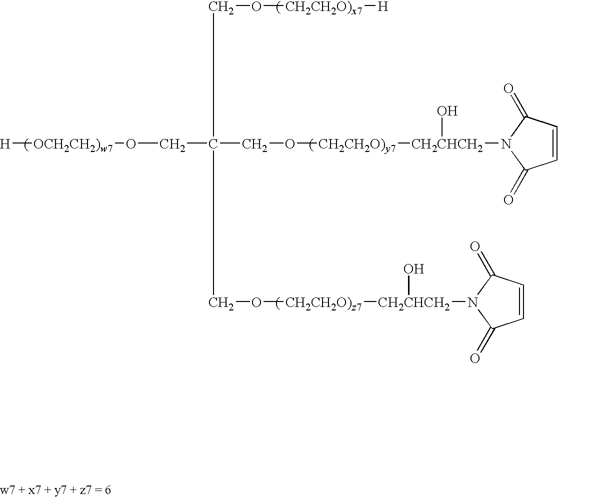

In the Exemplified Compound 7, Z of the general formula (I) corresponds to a pentaerythritol residue (shown below).

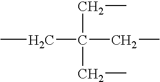

### Exemplified Compound 8

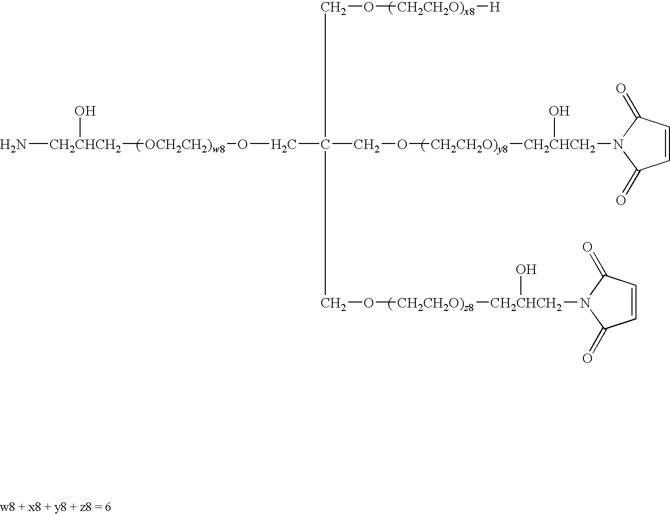

In the Exemplified Compound 8, Z of the general formula (I) corresponds to a pentaerythritol residue (shown below).

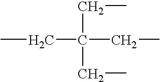

### Exemplified Compound 9

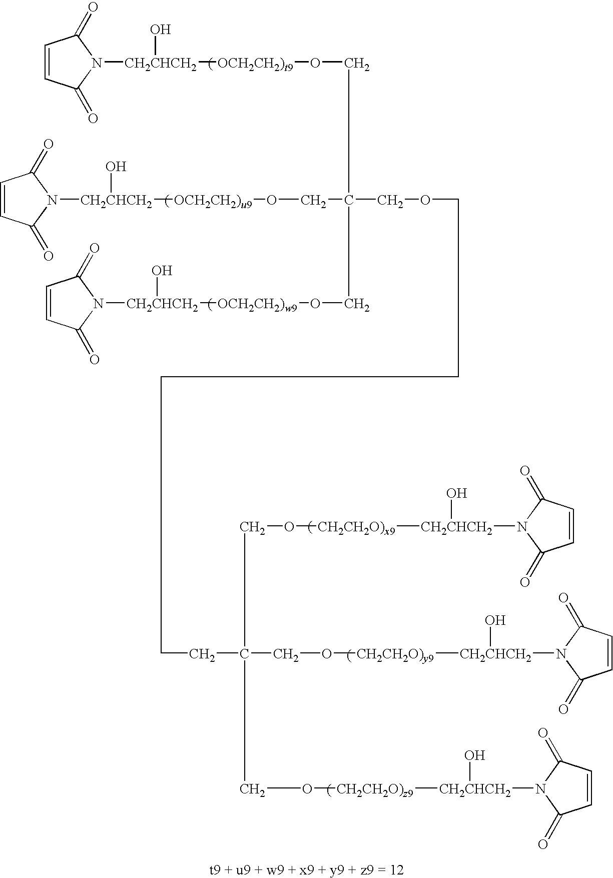

In the Exemplified Compound 9, Z of the general formula (I) corresponds to a dipentaerythritol residue (shown below).

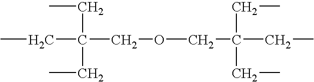

### Exemplified Compound 10

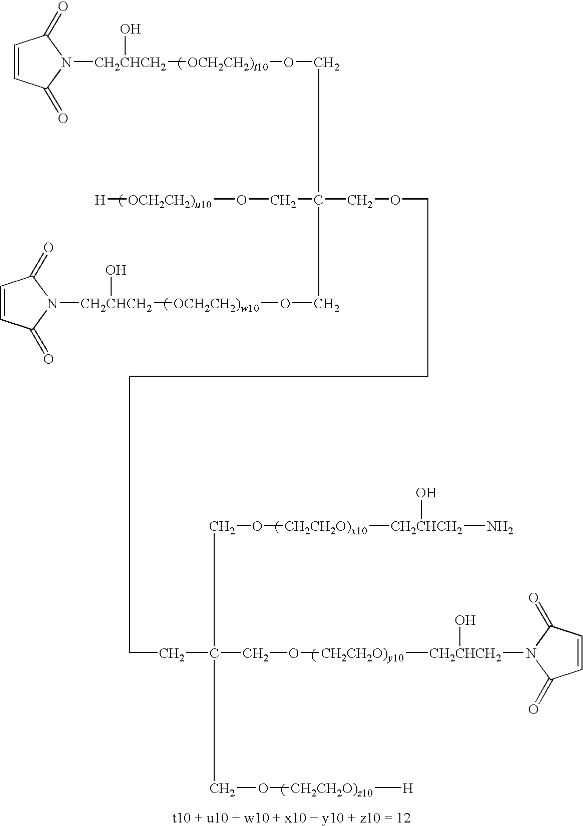

In the Exemplified Compound 10, Z of the general formula (I) corresponds to a dipentaerythritol residue (shown below).

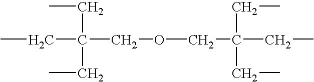

### Exemplified Compound 36

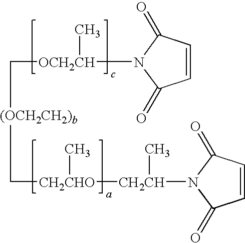

In the Exemplified Compound 36, Z of the general formula (I) corresponds to a polyethylene glycol residue having an, average molecular weight of about 400, represented by —(O—CH₂—CH₂)_{b}—. The average unit number b is about 9. R₁ and R₂ in A and B of the general formula (I) are each a methyl group, and the value of a+c is about 3.6.

### Exemplified Compound 37

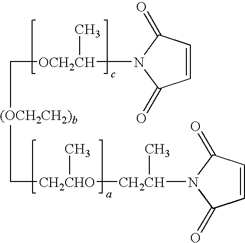

In the Exemplified Compound 37, Z of the general formula (I) corresponds to a polyethylene glycol residue having an average molecular weight of about 1,700, represented by —(O—CH₂—CH₂)_{b}—. The average unit number b is about 38.7. R₁ and R₂ in A and B of the general formula (I) are each a methyl group, and the value of a+c is about 6.

Of these, Exemplified Compounds 2, 5, 6 and 10 are particularly preferred. As other exemplary compounds, the following Exemplified Compounds 11 to 24, 38 and 39 may be cited in which the maleimide groups of the above Exemplified Compounds have been changed to itaconimide groups.

### Exemplified Compound 11

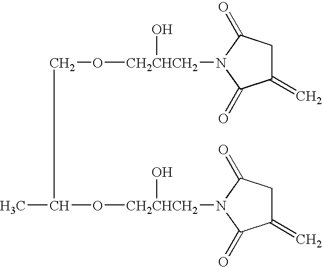

In the Exemplified Compound 11, Z of the general formula (I) corresponds to a propylene glycol residue (shown below).

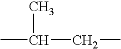

### Exemplified Compound 12

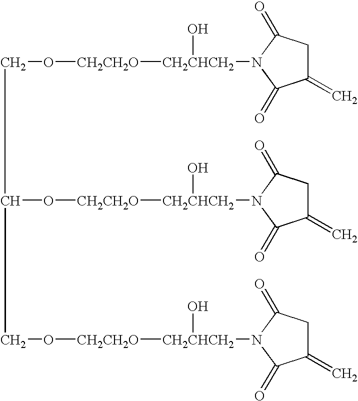

In the Exemplified Compound 12, Z of the general formula (I) corresponds to a glycerol residue (shown below).

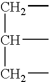

### Exemplified Compound 13

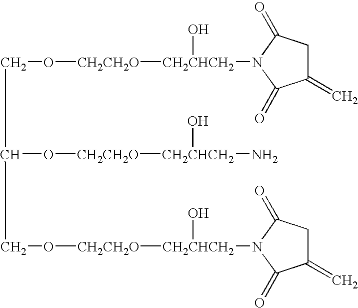

In the Exemplified Compound 13, Z of the general formula (I) corresponds to a glycerol residue (shown below).

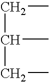

### Exemplified Compound 14

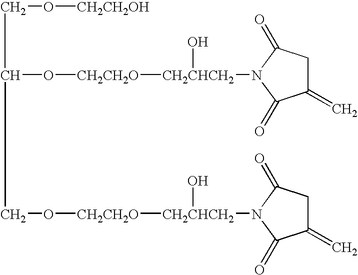

In the Exemplified Compound 14, Z of the general formula (I) corresponds to a glycerol residue, (shown below).

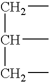

### Exemplified Compound 15

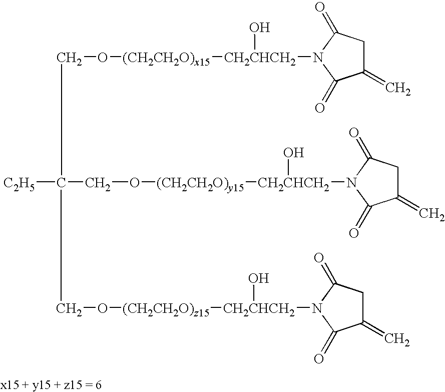

In the Exemplified Compound 15, Z of the general formula (I) corresponds to a trimethylolpropane residue (shown below).

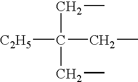

### Exemplified Compound 16

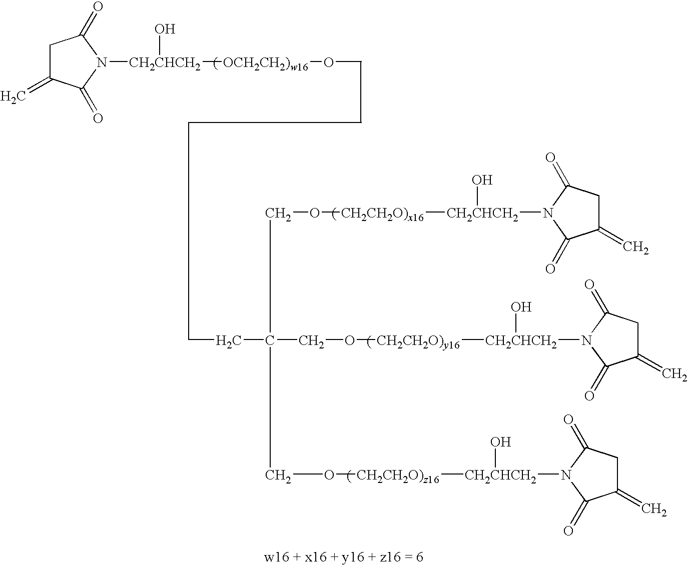

In the Exemplified Compound 16, Z of the general formula (I) corresponds to a pentaerythritol residue (shown below).

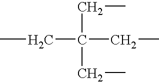

### Exemplified Compound 17

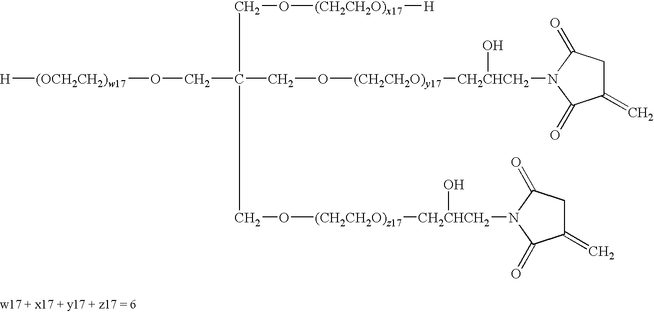

In the Exemplified Compound 17, Z of the general formula (I)-corresponds to a pentaerythritol residue (shown below).

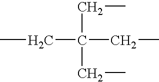

### Exemplified Compound 18

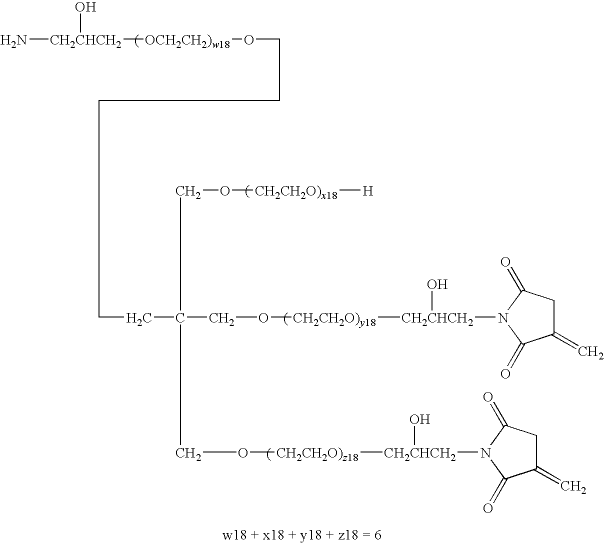

In the Exemplified Compound 18, Z of the general formula (I) corresponds to a pentaerythritol residue (shown below).

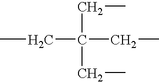

### Exemplified Compound 19

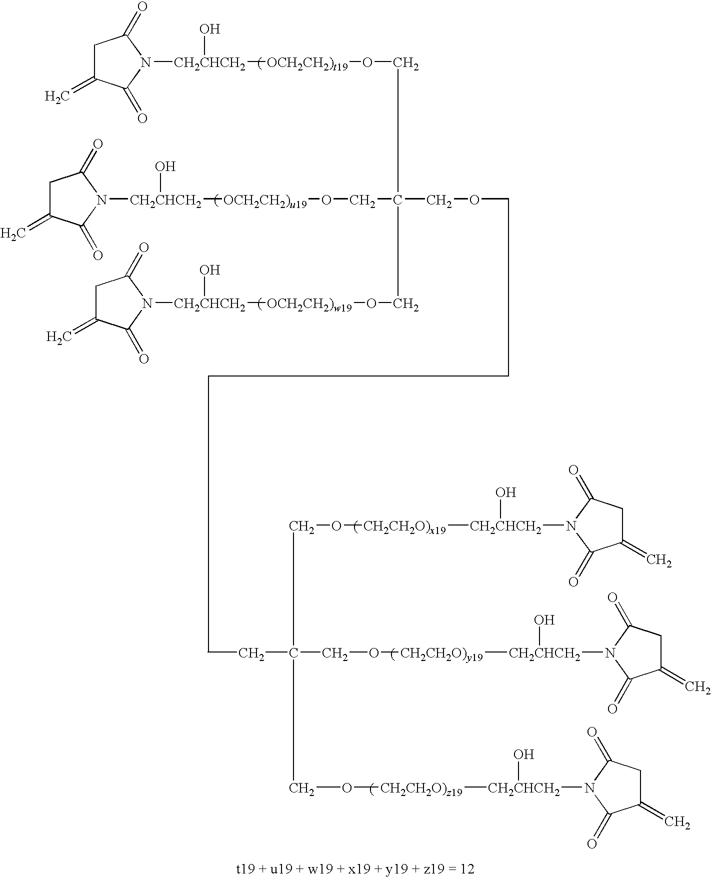

In the Exemplified Compound 19, Z of the general formula (I) corresponds to a dipentaerythritol residue (shown below).

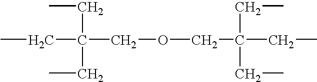

### Exemplified Compound 20

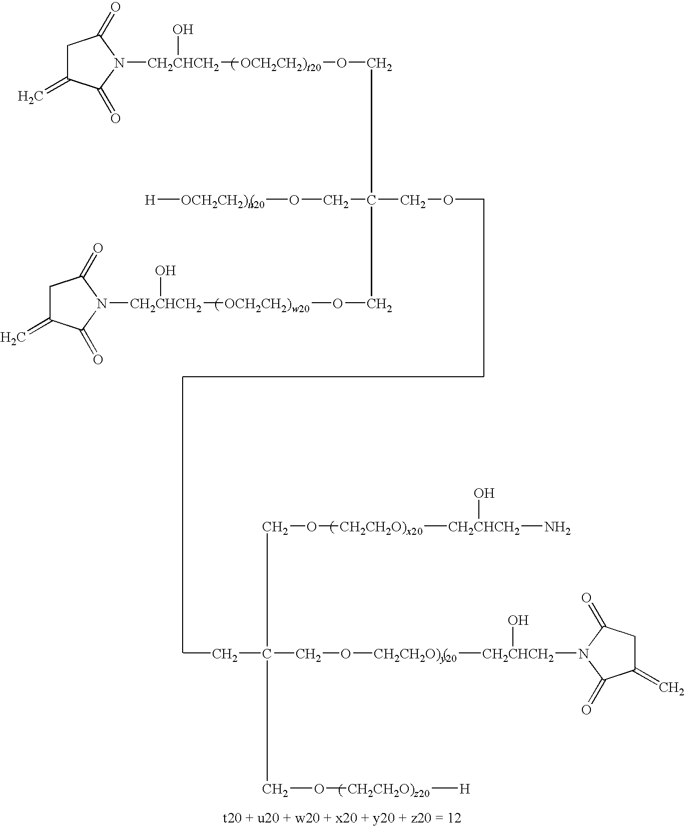

In the Exemplified Compound 20, Z of the general formula (I) corresponds to a dipentaerythritol residue (shown below).

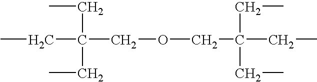

### Exemplified Compound 21

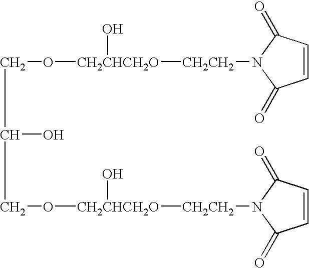

In the Exemplified Compound 21, Z of the general formula (I) corresponds to a glycerol residue (shown below).

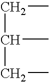

### Exemplified Compound 22

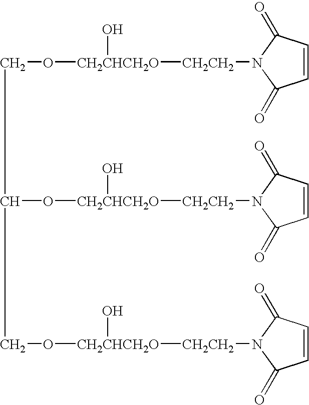

In the Exemplified Compound 22, Z of the general formula (I) corresponds to a glycerol residue (shown below).

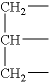

### Exemplified Compound 23

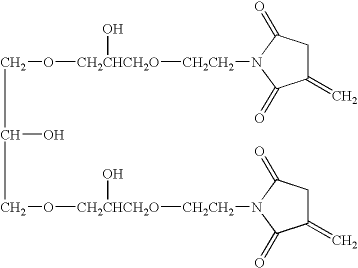

In the Exemplified Compound 23, Z of the general formula (I) corresponds to a glycerol residue (shown below).

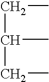

### Exemplified Compound 24

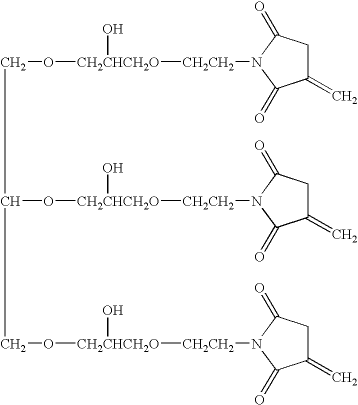

In the Exemplified Compound 24, Z of the general formula (I) corresponds to a glycerol residue (shown below).

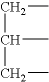

### Exemplified Compound 38

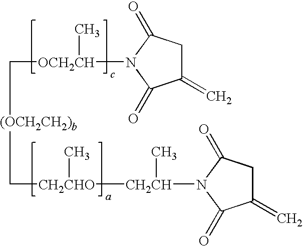

In the Exemplified Compound 38, Z of the general formula (I) corresponds to a polyethylene glycol residue having an average molecular weight of about 400, represented by —(O—CH₂—CH₂)_{b}—. The average unit number b is about 9. R₁ and R₂ in A and B of the general formula (I) are each a methyl group, and the value of a+c is about 3.6.

### Exemplified Compound 39

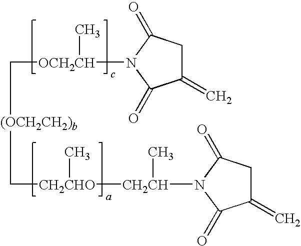

In the Exemplified Compound 39, Z of the general formula (I) corresponds to a polyethylene glycol residue having an average molecular weight of about 1,700, represented by —(O—CH₂—CH₂)_{b}—. The average unit number b is about 38.7. R₁ and R₂ in A and B of the general formula (I) are each a methyl group, and the value of a+c is about 6.

The active-energy radiation-polymerizable substance of the present invention, represented by the general formula (I), is produced by, e.g., a process as shown below. First, a compound having an epoxy group at the terminal is ring-opened with an amino group to prepare an amino compound. Next, the amino compound is allowed to react with maleic anhydride or itaconic anhydride to convert the terminal amino group into amic acid. Further, the amic acid is allowed to react with acetic anhydride. The desired imide compound can be obtained through such reaction. Of course, the process of producing the active-energy radiation polymerizable substance is by no means limited to this process.

—Active-Energy Radiation-Curable Liquid Composition, Active-Energy Radiation-Curable Ink—

—Polymerization Initiator—

The active-energy radiation-curable liquid composition and the active-energy radiation curable ink of the present invention contain the above-mentioned active-energy radiation-polymerizable substance, and may preferably contain a polymerization initiator. Such a polymerization initiator may preferably be hydrophilic. In the present invention, that “compound is hydrophilic” means that the compound is in any of the following states. (1) The compound is soluble in an organic solvent miscible with water, and the organic solvent is water-soluble. (2) Even if the compound is not water-soluble, it has been so treated as to be emulsifiable with water. (3) The compound is water-soluble.

Such a hydrophilic polymerization initiator used in the present invention may be any compound as long as being capable of generating a radical by the aid of an active-energy radiation. In the present invention, it is preferable to use at least one compound selected from the group consisting of compounds represented by the following general formulas (VI) and (VIII) to (XI).

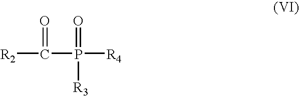

In the general formula (VI), R₂ is an alkyl group or an aryl group; R₃ is an alkyloxy group, a phenyl group or -OM; M is a hydrogen atom or an alkali metal; and R₄ is a group represented by the following general formula (VII).

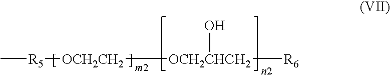

In the general formula (VII), R₅

is —[CH₂]ₓ₂— (where x2 is 0 to 1) or a phenylene group; m2 is 0 to 10; n2 is 0 to 1; and R₆ is a hydrogen atom, or a sulfonic acid group, a carboxyl group, a hydroxyl group, or a salt thereof.

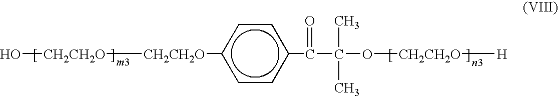

In the general formula (VIII), m3 is 1 or more, n3 is 0 or more, and m3+n3 is 1 to 8.

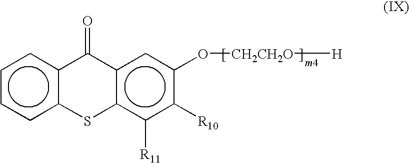

In the general formula (IX), R₁₀ and R₁₁ are each independently a hydrogen atom or an alkyl group, and m4 is 5 to 10.

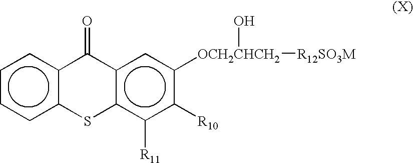

In the general formula (X), R₁₀ and R₁₁ are each independently a hydrogen atom or an alkyl group; R₁₂ is —(CH₂)ₓ (where x is 0 to 1), -0-(CH₂)_{y} (where y is 1 to 2) or a phenylene group; and M is a hydrogen atom or an alkali metal.

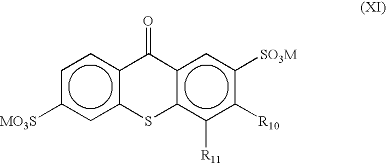

In the general formula (XI), R₁₀ and R₁₁ are each independently a hydrogen atom or an alkyl group, and M is a hydrogen atom or an alkali metal.

Of these, it is preferable to use the compounds represented by the general formulas (VI), (VIII) and (IX). It is particularly preferable to use the compounds represented by the general formulas (VI) and (VIII).

The alkyl group and aryl group represented by R₂ in the general formula (VI) may have a substituent. Such a substituent may include the following: A halogen atom, an alkyl group having 1 to 5 carbon atoms, an alkyloxy group having 1 to 5 carbon atoms, the group represented by the general formula (VII), a sulfonic acid group or a salt thereof, a carboxyl group or a salt thereof, and a hydroxyl group or a salt thereof. In the present invention, it is particularly preferable that R₂ is a aryl group having as the substituent the alkyl group having 1 to 5 carbon atoms. The counter ion that forms the salt of the sulfonic acid group, carboxyl group or hydroxyl group may preferably be the following: for example, an alkali metal, an alkaline earth metal or an ammonium group represented by HNR₇R₈R₉ (where R₇, R₈ and R₉ are each independently a hydrogen atom, an alkyl group having 1 to 5 carbon atoms, a monohydroxyl substituted alkyl group having 1 to 5 carbon atoms, or a phenyl group).

The phenylene group represented by R₅ in the general formula (VII) may have a substituent. Such a substituent may include the following: a halogen atom, an alkyl group having 1 to 5 carbon atoms, an alkyloxy group having 1 to 5 carbon atoms, a sulfonic acid group or a salt thereof, a carboxyl group or a salt thereof, and a hydroxyl group or a salt thereof. The counter ion that forms the salt of the sulfonic acid group, carboxyl group or hydroxyl group may preferably be the following: for example, an alkali metal, an alkaline earth metal or an ammonium group represented by HNR₇R₈R₉ (where R₇, R₈ and R₉ are each independently a hydrogen atom, an alkyl group having 1 to 0.5 carbon atoms, a monohydroxyl substituted alkyl group having 1 to 5 carbon atoms, or a phenyl group).

R₆ in the general formula (VII) is, as defined above, a hydrogen atom, a sulfonic acid group or a salt thereof, a carboxyl group or a salt thereof, or a hydroxyl group or a salt thereof. The counter ion that forms the salt of the sulfonic acid group, carboxyl group or hydroxyl group may preferably be the following: for example, an alkali metal, an alkaline earth metal or an ammonium group represented by HNR₇R₈R₉ (where R₇, R₈ and R₉ are each independently a hydrogen atom, an alkyl group having 1 to 5 carbon atoms, a monohydroxyl substituted alkyl group having 1 to 5 carbon atoms, or a phenyl group).

The alkyloxy group and phenyl group represented by R₃ in the general formula (VI) may have a substituent. Such a substituent may include, e.g., the following: a halogen atom, an alkyl group having 1 to 5 carbon atoms and an alkyloxy group having 1 to 5 carbon atoms. Particularly preferred R₃ is an alkyloxy group, in particular, —OC₂H₅ or —OC(CH₃)₃.

The alkyl group represented by each of R₁₀ and R₁₁ in the general formula (X) may have a substituent. Such a substituent may include, e.g., the following: a halogen atom, a sulfonic acid group or a salt thereof, a carboxyl group or a salt thereof, and a hydroxyl group or a salt thereof. The counter ion that forms the salt of the sulfonic acid group, carboxyl group or hydroxyl group may preferably be the following: for example, an alkali metal, an alkaline earth metal or an ammonium group represented by HNR₇R₈R₉ (where R₇; R₈ and R₉ are each independently a hydrogen atom, an alkyl group having 1 to 5 carbon atoms, a monohydroxyl substituted alkyl group having 1 to carbon atoms, or a phenyl group).

In the general formulas (VI) to (XI), the alkyl group may preferably be a straight-chain or branched alkyl group having 1 to 5 carbon atoms, which may specifically include a methyl group, an ethyl group, a propyl group, a butyl group and a pentyl group. The alkyloxy group may preferably be a straight-chain or branched alkyloxy group having 1 to 5 carbon atoms, which may specifically include a methoxy group, an ethoxy group, a propoxy group, a butoxy group and a pentoxy group. As specific examples of the alkali metal, it may include lithium, sodium and potassium. As specific examples of the alkaline earth metal, it may include calcium, strontium and barium. As specific examples of the ammonium group represented by HNR₇R₈R₉, it may include ammonium, dimethylethanolammonium, methyldiethanolammonium, triethanolammonium and anilinium. Of course, in the present invention, examples are by no means limited to these.

As particularly preferred examples of the polymerization initiator usable in the present invention, it may include those having structures shown below. Of course, in the present invention, examples are by no means limited to these.

### Exemplified Compound 25

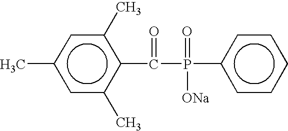

### Exemplified Compound 26

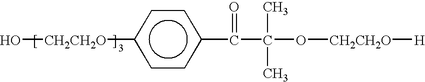

### Exemplified Compound 27

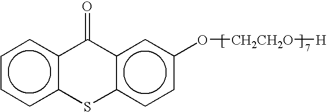

### Exemplified Compound 28

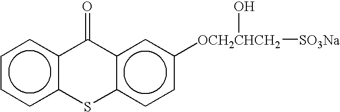

### Exemplified Compound 29

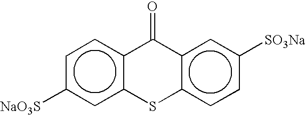

When the active-energy radiation-polymerizable substance of the present invention is used in the liquid composition or the ink, the liquid composition or the ink may preferably be constituted in the following way. In order to improve radical generation efficiency of the polymerization initiator, a hydrogen-donating agent such as triethanolamine or monoethanolamine may preferably be used in combination with the polymerizable substance. The hydrogen-donating agent such as triethanolamine or monoethanolamine may preferably be used in combination especially when a thioxanthone-type polymerization initiator or the like is used as the polymerization initiator. The hydrogen-donating agent in the liquid composition or the ink may preferably be in a content of 0.5% by mass or more and 30% by mass or less based on the content of the active-energy radiation-polymerizable substance. Of course, the hydrogen-donating agent usable in the present invention is by no means limited to these.

In the present invention, two or more types of polymerization initiators may be used in combination. When using two, or more types of polymerization initiators in combination, the radicals are expected to be more generated by utilizing light having wavelengths not effectively utilizable when one type of polymerization initiator is used. The polymerization initiator as described above is not necessarily required to be used when an electron ray curing method is employed in which electron rays are used as the active-energy radiation to cure the liquid composition or the ink.

—Coloring Material—

The active-energy radiation-polymerizable substance of the present invention is used in the ink containing a coloring material, whereby the ink is utilizable as a colored active-energy radiation-curable ink which can be cured by irradiation with an active-energy radiation. The active-energy radiation-curable ink of the present invention contains at least an active-energy radiation-polymerizable substance and coloring material, it is preferable to use as the coloring material a pigment dispersion in which a pigment has uniformly been dispersed in an aqueous medium. As this pigment dispersion, it is particularly preferable to use a pigment dispersion in which a pigment has stably been dispersed in an aqueous medium in virtue of an anionic group. In addition, it is possible to use, e.g., a pigment, dispersion for aqueous gravure inks or for aqueous writing utensils which is stable in a nonionic or anionic condition, and a pigment dispersion used in conventionally known ink jet recording inks.

A pigment dispersion in which a pigment has been dispersed by using a water-soluble high polymer having an anionic group and being alkali-soluble is disclosed in, e.g., Japanese Patent Applications Laid-open No. H05-247392 and No. H08-143802. A pigment dispersion in which a pigment has been dispersed by using a surface-active agent having an anionic group is disclosed in Japanese Patent Applications Laid-open No. H08-209048. Pigment dispersions in which a pigment has been dispersed by using pigment particles micro-encapsulated with a high polymer and provided on the capsule surfaces with anionic groups are disclosed in the following publications: for example, Japanese Patent Applications Laid-open No. H10-140065, No. H09-316353, No. H09-151342, No. H09-104834, and No. H09-031360. Further, pigment dispersions in which a pigment has been dispersed by using pigment particles to the surfaces of which anionic groups have been bonded by chemical reaction are disclosed in U.S. Pat. No. 5,837,045 and No. 5,851,280. In the ink of the present invention, any of various pigment dispersions as described above may be used as the coloring material of the ink.

The ink of the present invention is not limited to an embodiment in which the above pigment is used, and may be realized as an embodiment in which a water-soluble dye used as the coloring material is contained in a dissolved state. This is also possible as long as no problem is raised in discoloration due to irradiation with an active-energy radiation does not come into question in practical use. A coloring material dispersion containing a disperse dye, an oil-soluble dye or the like in a dispersed state may also be used as in the above pigment dispersion. These may appropriately be selected according to uses.

In the case where a pigment is used as the coloring material of the ink of the present invention, it is preferable to use a pigment dispersion in which the pigment has been dispersed in the form of fine particles. In particular, a pigment dispersion preferably usable in the ink is preferably provided with the following fundamental factors. Specifically, it is preferable that the pigment is dispersed in an aqueous medium and has a particle size distribution as a pigment dispersion in the range of 25 nm or more and 350 nm or less in average particle diameter and that the viscosity of the ink containing such a pigment dispersion is controllable within the range in which the ejection of ink performed in an ink jet recording system is not affected. It is further required for the pigment dispersion to satisfy the compatibility with the above active-energy radiation-polymerizable substance of the present invention which is essential for making the ink curable with an active-energy radiation.

(Pigment)

The pigment usable in the ink of the present invention may include carbon black and organic pigments. The pigment in the ink may preferably be in a content of 0.3% by mass or more and 10.0% by mass or less based, on the total mass of the ink.

The carbon black may include furnace black, lamp black, acetylene black and channel black, which may preferably have the following characteristics: a primary particle diameter of 15 nm or more and 40 nm or less, a specific surface area of 50 m²/g or more and 300 m²/g or less as measured by BET method, a DBP oil absorption of 40 ml/100 g or more and 150 ml/100 g or less, a volatile content of 0.5% or more and 10% or less and a pH value of 2 or more and 9 or less. In the present invention, the following carbon blacks may be used as commercially available products having the above characteristics.

RAVEN 7000, RAVEN 5750, RAVEN 5250, RAVEN 5000, RAVEN 3500, RAVEN 2000, RAVEN 1500, RAVEN 1250, RAVEN 1200, RAVEN 1190 ULTRA-II, RAVEN 1170 and RAVEN 1255 (the foregoing are available from Columbian Carbon Japan Limited); BLACK PEARLS L, REGAL: 400R, REGAL 330R, REGAL 660R, MOGUL L, MONARCH 700, MONARCH 800, MONARCH 880, MONARCH 900, MONARCH 1000, MONARCH 1100, MONARCH 1300, MONARCH 1400 and VALCAN XC-72R (the foregoing are available from Cabot Corp.); COLOR BLACK FW1, COLOR BLACK FW2, COLOR BLACK FW2V, COLOR BLACK FW18, COLOR BLACK FW200, COLOR BLACK S150, COLOR BLACK S160, COLOR BLACK S170, PRINTEX 35, PRINTEX U, PRINTEX V, PRINTEX 140U, PRINTEX 140V, SPECIAL BLACK 6, SPECIAL BLACK 5, SPECIAL BLACK 4A and SPECIAL BLACK 4 (the foregoing are available from Degussa Corp.); and No. 25, No. 33, No. 40, No. 47, No. 52, No. 900, No. 2300; MCF-88, MA600, MA7, MA8 and MA100, (the foregoing are available from Mitsubishi Chemical Corporation). Of course, besides these, any conventionally known carbon black may be used. Magnetic fine particles of magnetite, ferrite or the like and titanium black may also be used as the pigment.

As the organic pigment, specifically, the following may be used.

Water-insoluble azo pigments such as Toluidine Red, Toluidine maroon, Hanza Yellow, Benzidine Yellow and Pyrazolone Red; water-soluble azo pigments such as Lithol Red, Helio Bordeaux, Pigment Scarlet and Permanent Red 2B; derivatives of vat dyes, such as alizarin, indanthrone and Thioindigo maroon; phthalocyanine pigments such as Phthalocyanine Blue and Phthalocyanine Green; quinacridone pigments such as Quinacridone Red and Quinacridone Magenta; perylene pigments such as Perylene Red and Perylene Scarlet; isoindolinone pigments such as Isoindolinone Yellow and Isoindolinone Orange; imidazolone pigments such as Benzimidazolone Yellow, Benzimidazolone Orange and Benzimidazolone Red; pyranthrone pigments such as Pyranthrone Red and Pyranthrone Orange; indigo pigments, thioindigo pigments and condensation azo pigments; and Flavanthrone Yellow, Acyl Amide Yellow, Quinophthalone Yellow, Nickel Azo Yellow, Copper Azomethine Yellow, Perinone Orange, Anthrone Orange, Dianthraquinonyl Red and Dioxazine Violet.

Where the organic pigments are shown by Color Index (C.I.) Number, the following may be used.

### C.I. Pigment Yellow 12, 13, 14, 17, 20, 24, 55, 74, 83, 86, 93, 97, 98, 109, 110, 117, 120, 125, 128, 137, 138, 139, etc.; and C.I. Pigment Yellow 147, 148, 150, 151, 153, 154, 155, 166, 168, 180, 185, etc.;

### C.I. Pigment Orange 16, 36, 43, 51, 55, 59, 61, 71, etc.;

### C.I. Pigment Red 9, 48, 49, 52, 53, 57, 97, 122, 123, 149, 168, 175, 176, 177, 180, 192, 202, 209, 215, 216, 217, etc.; and C.I. Pigment Red 220, 223, 224, 226, 227, 228, 238, 240, 254, 255, 272, etc.;

### C.I. Pigment Violet 19, 23, 29, 30, 37, 40, 50, etc.;

### C.I. Pigment Blue 15, 15:1, 15:3, 15:4, 15:6, 22, 60, 64, etc.;

### C.I. Pigment Green 7, 36, etc.; and

### C.I. Pigment Brown 23, 25, 26, etc.

(Resin Dispersion Pigment)

In the case where the above carbon black or organic pigments are used, it is preferable to use a dispersant (a resin acting as a dispersant) in combination to disperse the pigment. As the dispersant, it is preferable to use what can stably disperse carbon black or organic pigments in an aqueous medium by the action of anionic groups.

(Self-Dispersion Pigment)

In the case where the above carbon black or organic pigments are used, it is possible to use what is called a self-dispersion pigment in which ionic groups (e.g., anionic groups) are bonded to the surfaces of pigment particles so that the pigment particles can disperse into an aqueous medium without use of any dispersant.

(Dispersant)

As the dispersant, it is preferable to use what can stably disperse carbon black or organic pigments in an aqueous medium by the action of anionic groups. As the dispersant, a block polymer, a random polymer, a graft polymer or the like may be used, which may specifically include, e.g., the following: A styrene-acrylic acid copolymer, styrene-acrylic acid-alkyl acrylate copolymers, a styrene-maleic acid copolymer, and styrene-maleic acid-alkyl acrylate copolymers, or salts thereof; a styrene-methacylic acid copolymer, and styrene-methacrylic acid-alkyl acrylate copolymers, or salts thereof; a styrene-maleic half ester copolymer, a vinyl naphthalene-acrylic acid copolymer, a vinyl naphthalene-maleic acid copolymer, and a styrene-maleic anhydride-maleic half ester copolymer, or salts thereof; and a benzyl methacrylate-methacrylic acid copolymer, or salts thereof.

(Particle Diameter of Pigment)

The pigment may preferably have an average particle diameter of 25 nm or more and 350 nm or less, and more preferably 70 nm or more and 200 nm or less. As long as the average particle diameter of the pigment is within the above range, it is sufficiently smaller than the wavelength of visible light, and hence recorded matter which can be said to be sufficiently transparent can be obtained if light scattering is small, though depending on what the recorded matter is used for.

(Dye)

The ink of the present invention may preferably be irradiated with an active-energy radiation after the ink has been applied to the recording medium, to polymerize the active-energy radiation polymerizable substance in the ink to effect curing. In the case where a dye is used as described previously, differently from the case in which a pigment is used, it is difficult to use a dye in the state of being entirely free of any discoloration due to irradiation with an active-energy radiation, and such discoloration some what occurs. For this reason, in the case where a dye is used as the coloring material of the ink, what is called an azo-containing dye, in which a complex is formed by the union of a metal ion with a ligand, may be used. This is preferable because discoloration due to light is reduced. However, if the level of discoloration is not taken into account, at least some inks can be made up even using common water-soluble dyes.

Supposing the above, where dyes are shown by Color Index (C.I.) Number, the following may be used.

### C.I. Acid Yellow 11, 17, 23, 25, 29, 42, 49, 61, 71, etc.;

### C.I. Direct Yellow 12, 24, 26, 44, 86, 87, 98, 100, 130, 132, 142, etc.;

### C.I. Acid Red 1, 6, 8, 32, 35, 37, 51, 52, 80, 85, 87, 92, 94, 115, 180, 254, 256, 289, 315, 317, etc.;

### C.I. Direct Red 1, 4, 13, 17, 23, 28, 31, 62, 79, 81, 83, 89, 227, 240, 242, 243, etc.;

### C.I. Acid Blue 9, 22, 40, 59, 93, 102, 104, 113, 117, 120, 167, 229, 234, 254, etc.;

### C.I. Direct Blue 6, 22, 25, 71, 78, 86, 90, 106, 199, etc.; and

### C.I. Direct Black 7, 19, 51, 154, 174, 195, etc.

The dye in the ink may preferably be in a content of 0.1% by mass or more and 10% by mass or less based on the total mass of the ink. Where the dye is in a small content, it is used in, e.g., what is called light-color inks.

—Constitution in Making Up Liquid Composition—

The ink of the present invention may be in the form of a transparent ink without containing the above coloring material, so as to be an active-energy radiation-curable liquid composition (hereinafter referred to simply as “liquid composition” in some cases). The use of this liquid composition enables a substantially colorless and transparent film to be formed, because it contains no coloring material. Such a liquid composition may be used for the following purposes. For example, it may be used for undercoats which are formed to provide recording mediums with suitability to image recording, for the surface protection of images formed using usual inks, and for overcoats intended for decoration, impartation of gloss and so forth. The liquid composition may contain a colorless pigment, colorless fine particles or the like dispersed therein, not intended for coloring, in accordance with uses such as prevention of oxidation and prevention of discoloration. When adding these, it is possible to improve various properties or characteristics such as image quality, fastness and processability (handling properties) of recorded matter, in any of the undercoats and the overcoats.

When used in such a liquid composition, the liquid composition may preferably be so made up that the active-energy radiation-polymerizable substance is in a content of 10% by mass or more and 70% by mass or less based on the total mass of the liquid composition. The polymerization initiator may preferably be in a content of 1 part by mass or more and 10 parts by mass or less based on 100 parts by mass of the polymerizable substance. At the same time, the polymerization initiator may preferably be in a content of 0.5% by mass or more based on the total mass of the liquid composition. The aqueous medium (water or an organic solvent, or a mixture of water and an organic solvent) may preferably be in a content of 10% by mass or more and 90% by mass or less based on the total mass of the liquid composition.

—Reactive Diluent—

In the liquid composition of the present invention, a polymerizable low-viscosity monomer may be contained as a reactive diluent. An advantage in using not a usual organic solvent but such a substance is as follows: This substance by no means remains as a plasticizer in the solid cured with an active-energy radiation. Hence, an influence as a plasticizer on the physical properties of the solid is reduced. The reactive diluent used for such purpose may specifically include, e.g., the following: Acryloyl morpholine, N-vinylpyrrolidone, acrylamide, methylenebisacrylamide, monoacrylates of monosaccharides, monoacrylates of oligoethylene oxides, and monoacrylates of dibasic acids.

—Organic Solvent—

In the liquid composition of the present invention, it is particularly preferable not to use organic solvents capable of giving moisture retention, such as used conventionally in aqueous ink jet recording inks. This is because the liquid composition does not contain any solid component such as pigment, hence the thickening of the liquid composition is so small as to be readily restorable even if it has somewhat thickened. Of course, organic solvents having a higher moisture retention as described later may be added in a necessary and minimum quantity. These may appropriately be selected from a large number of compounds having conventionally been in wide use in aqueous ink jet recording inks.

—Constitution in Making Up Ink—

In the case where the liquid composition of the present invention is used as an ink containing the coloring material, an organic solvent may be added to the ink. The organic solvent is added for the purposes of, e.g., providing the ink with non-volatility, reducing the viscosity of the ink and providing the ink with wettability to recording mediums. In the case of recording on non-absorptive recording mediums, the ink may preferably be so made up as to contain no organic solvent and contain only water so that the polymerizable substance may entirely cure to become solid.

Where the organic solvent is added to the ink in an amount of 10% by mass or more, the recording medium may have a certain absorptivity. This is preferable from the viewpoint of the strength of ink layers obtained finally. For example, in the case of recording using an aqueous gravure ink, a recording medium provided with certain wettability and permeability is used, and forced-drying is carried out. As in this case, in the ink of the present invention as well, it is preferable that when the organic solvent is added to the ink in an amount of 10% by mass or more, the recording medium is subjected to pretreatment to be provided with ink receptivity, and subjected to natural or forced drying after the ink has been cured with an active-energy radiation. The active-energy radiation-polymerizable substance of the present invention has a certain moisture retention in itself (to keep water from evaporating and to absorb water), and hence the ink may be so made up that the organic solvent is completely extruded. In such a case, measures such as capping, suction of ink at the start of recording and preliminary ejection may be taken in order to secure the reliability of an ink jet recording apparatus on the level of practical use.

Organic solvents are enumerated below which evaporate to dryness relatively with ease and are usable in the ink of the present invention. In the present invention, what has arbitrarily been selected from these organic solvents may be added. Glycol ethers such as ethylene glycol monomethyl ether, ethylene glycol monoethyl ether, ethylene glycol isopropyl ether, and ethylene glycol monoallyl ether; glycol ethers such as diethylene glycol monomethyl ether, and diethylene glycol monoethyl ether; glycol ethers such as triethylene glycol monomethyl ether, triethylene glycol monoethyl ether, propylene glycol monomethyl ether, and dipropylene glycol monomethyl ether; and monohydroxyl alcohols such as methanol, ethanol, propanol, butanol and pentanol.

In the case where the liquid composition of the present invention is used as the ink containing the coloring material, the content of the polymerization initiator and the content of the active-energy radiation-polymerizable substance in the ink may preferably be controlled in accordance with absorption characteristics of the coloring material. The aqueous medium (water or an organic solvent, or a mixture of water and an organic solvent) may preferably be in a total content of 30% by mass or more and 90% by mass or less based on the total mass of the ink. The active-energy radiation-polymerizable substance may preferably be in a content of 1% by mass or more and 35% by mass or less, and more preferably 10% by mass or more and 25% by mass or less, based on the total mass of the ink. The polymerization initiator may preferably be in a content, which depends on the content of the active-energy radiation-polymerizable substance, of approximately 0.1% by mass or more and 7% by mass or less, and more preferably 0.3% by mass or more and 5% by mass or less, based on the total mass of the ink.

In the case where the pigment is used as the coloring material of the ink, the pigment in the ink may preferably be in a content of 0.3% by mass or more and 10% by mass or less based on the total mass of the ink. While the coloring power of the pigment depends on the dispersion state of pigment particles, when the pigment is in a content of 0.3% by mass or more and 1% by mass or less, it is in a range in which the ink is used as what is called light-color ink. When the pigment is in a content of more than that range, it is in a range in which the ink is used as dark-color ink for common color recording. The content of the pigment dispersion also depends on the viscosity and flow properties of ink that are suited for ink jet recording apparatus.

Where the ink of the present invention is used in an on-demand type ink jet recording method, the ink may preferably have the viscosity upper limit of 15 mPa·s at 25° C. Where the ink of the present invention is used in an ink jet recording apparatus having nozzles with a high density and a high drive frequency, the ink may preferably have the viscosity upper limit of 10 mPa·s at 25° C.

Taking into account the fact that the ink of the present invention is used to make records on recording mediums such as plain paper, the ink may preferably have a surface tension of 35 mN/m (dyne/cm) or more at 25° C. In the recording on plain paper, it is preferable to prevent coloring materials from bleeding between them. Accordingly, in usual inks for ink jet recording, the surface tension must be controlled to be as low as about 30 mN/m so that inks can permeate into the recording medium in a short time. In such a case, however, a decrease in image density may concurrently be brought about.

In contrast, the surface tension of the ink of the present invention may preferably be set to be higher so that the ink can stay on the surface of the recording medium as much as possible at the time of the irradiation with an active-energy radiation. In this way, the ink can effectively be cured in the vicinity of the surface of the recording medium, so that the bleeding can be prevented, and at the same time, a high image density can be achieved. In order to secure this image density, it is preferable for the ink to wet the recording medium to a certain extent at the time of the irradiation with an active-energy radiation. Accordingly, the ink of the present invention may more preferably have the surface tension upper limit of about 50 mN/m at 25° C.

—Ink Jet Recording Method, Ink Cartridge, Recording Unit, and Ink Jet Recording Apparatus—

The liquid composition or the ink of the present invention may preferably be used in a recording head of an ink jet recording system. The ink of the present invention is also effective as an ink stored in an ink cartridge or recording unit having an ink storage portion which stores the ink therein and also as an ink with which the ink cartridge is to be replenished. In particular, the ink of the present invention exhibits excellent effects in a recording head and an ink jet recording apparatus which are of an ink jet recording system, in particular, a system in which the ink is ejected by the action of thermal energy.

In respect of the typical construction and principles, a system is preferred in which recording is performed by the use of basic principles disclosed in, e.g., U.S. Pat. No. 4,723,129 and No. 4,740,796. This system is applicable to any of what are called an on-demand type and a continuous type. In particular, in the case of the on-demand type, this system is effective because at least one drive signal corresponding to recording information and calling for a rapid temperature rise that exceeds nucleate boiling is applied to an electricity-heat converter disposed correspondingly to a sheet or liquid channels on or through which the ink is stored, to generate thermal energy in the electricity-heat converter to bring about film boiling on the heating portion surface of a recording head, and consequently bubbles in ink can be formed in one to one correspondence to this drive signal. The growth and shrinkage of the bubbles cause the ink to eject through ejection orifices to form at least one droplet. Where this drive signal is applied in the form of a pulse, the growth and shrinkage of the bubbles take place instantly and appropriately, and hence the ejection of ink excellent especially in response can be achieved, thus this is more preferred. As this drive signal in pulse form, it is preferred to adopt what are disclosed in U.S. Pat. No. 4,463,359 and U.S. Pat. No. 4,345,262. When employing the conditions disclosed in U.S. Pat. No. 4,313,124, which relates to the rate of temperature rise on the heating portion surface, further superior recording can be performed.

The construction of the recording head may preferably be set up by combination of an ejection orifice, a liquid channel and an electricity-heat converter as disclosed in the above respective U.S. patents (a linear liquid channel or a right-angle liquid channel), and besides, be so set up that a heat build-up part is disposed in a bent region. These are, disclosed in U.S. Pat. No. 4,558,333 and U.S. Pat. No. 4,459,600. The present invention is effective also in the atmosphere communication type ejection system disclosed in Japanese Patents No. 2962880 and No. 3246949 and further in Japanese Patent Application Laid-open No. H11-188870. In addition, the present invention is effective also in a construction in which an ejection orifice common to a plurality of electricity-heat converters is provided as an ejection part of the electricity-heat converters (see Japanese Patent Application Laid-open No. S59-123670, etc.).

As a recording head of a full-line type, having a length corresponding to the width of a maximum recording medium on which an ink jet recording apparatus can perform recording, what is shown below may be used. For example, it may be so set up that its length condition is fulfilled by a combination of a plurality of recording heads as disclosed in the above publications, or may be so set up as to be one recording head which is integrally formed. In any recording heads set up as described above, the present invention can effectively bring out the above effect.

The present invention is effective also in an exchangeable chip type recording head in which, when set in an ink jet recording apparatus, electrical connection with the recording apparatus is established and ink is fed from the recording apparatus, or in a cartridge type recording head provided integrally in the recording head.

A restoration means, a preliminary auxiliary means and so forth for the recording head may be added which are provided to set up the ink jet recording apparatus. This is preferable because the effects of the present invention can be further stably exhibited. To give examples of these specifically, they are a capping means, a cleaning means and a pressure or suction means which are provided for the recording head; an electricity-heat converter or a heating means different therefrom, or a preliminary heating means set up by combination of these; and a preliminary ejection mode which performs ejection different from that for recording.

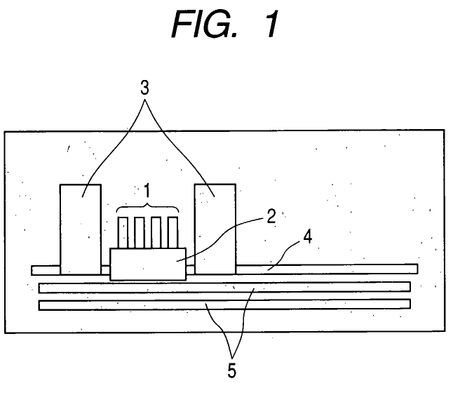

An ink jet recording apparatus is specifically described with reference to FIG. 1 which is a schematic front view of the apparatus. The ink jet recording apparatus has an ink cartridge 1 storing therein the ink, a recording head 2 which performs recording, lamps 3 which perform irradiation with an active-energy radiation for curing, a drive 4 which drives the recording head and lamps, and a paper delivery means 5 which transports recording mediums. The recording head 2 employs a multiple head in which recording heads are, arranged in a large number. Besides these, the apparatus has a wiping means, a capping means, a paper feed means and a drive motor (which are not shown in the FIGURE).

In FIG. 1, in the recording head 2, nozzles for ejecting ink are symmetrically arranged for each color. Then, the recording head 2 and the lamps 3 are moved together from side to side to apply inks to the recording medium, and thereafter the recording medium is immediately irradiated with an active-energy radiation. Thus, the inks can be prevented from spreading and from bleeding therebetween, and high-grade and highly colorful images can be obtained. An ultraviolet irradiation lamp is detailed later which is preferably usable as a source for an active-energy radiation.

In the ink cartridge 1, units for four colors, black (Bk), cyan (C), magenta (M) and yellow (Y), are disposed. Instead, units for six colors additionally including light cyan (LC) and light magenta (LM) may be disposed in order to record more highly colorful images. Since black ink has reactivity inferior to other inks, three-color disposition may also be available which has cyan, magenta and yellow in combination to form process black. In the present invention, it is preferable to use an ink cartridge that can shield light rays.

In the present invention, besides the ink jet recording apparatus described above; apparatuses may appropriately be selected which are exemplified by an apparatus the lamps of which are disposed in front of the paper delivery means, an apparatus in which paper feed and paper delivery are performed in the state the paper is wound around a rotating-drum, and an apparatus provided additionally with a drying means.

—Ultraviolet Irradiation Lamp—

The ultraviolet irradiation lamp used to cure the ink is described below, which is particularly preferable in the present invention. The ultraviolet irradiation lamp may preferably be, e.g., what is called a low-pressure mercury lamp, a high-pressure mercury lamp, or a mercury lamp coated with fluorescent material, having a mercury vapor pressure of 1 Pa or more and 10 Pa or less during lighting. These mercury lamps have emission spectra in the range of 184 nm or more and 450 nm or less in the ultraviolet region, which are suited to allow the polymerizable substance in black or colored ink to react efficiently. These enable a small-sized power source to be used in mounting a power source in the ink jet recording apparatus, and hence are suited in that sense as well. The mercury lamp includes, e.g., metal halide lamps, high-pressure mercury lamps, ultrahigh-pressure mercury lamps, xenon flash lamps, deep ultraviolet lamps, lamps in which mercury lamps are excited without electrodes from the outside by using microwaves, and ultraviolet lasers; which have been put into practical use. Emission spectra of these lamps are included in the above range, and hence these are basically applicable as long as the power source size, input intensity, lamp shape and so forth are acceptable. As to the light source, it may be selected in accordance with the sensitivity of the polymerization initiator to be used.

The ultraviolet rays usable to cure the ink of the present invention may preferably have an intensity of 500 mW/cm² or more and 5,000 mW/cm² or less in wavelength region effective in curing. At low irradiation intensity, the effects of the present invention may be not sufficiently obtained. On the other hand, at too high irradiation intensity, the recording medium may be damaged or coloring materials may be discolored.

### EXAMPLES

The present invention is described below in greater detail by giving synthesis examples of the active-energy radiation-polymerizable substance, examples and comparative examples of the liquid compositions and the inks. The following working examples should not be construed to limit the scope of the present invention. In the following, the amount of each component in the liquid compositions or the inks refers to “part(s) by mass” unless otherwise specified.

### Synthesis Example 1

### Synthesis of Exemplified Compound 30

### Exemplified Compound 30

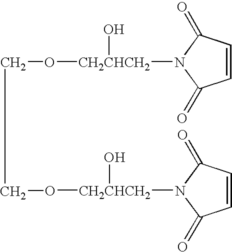

In the Exemplified Compound 30, Z of the general formula (I) corresponds to an ethylene glycol residue (shown below).

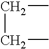

(a) Synthesis of Diamino Compound:

Into a solution in which 200 g (1.15 mols) of ethylene glycol diglycidyl ether (available from Tokyo Chemical Industry Co., Ltd.) was dissolved in 1 kg of ethanol, ammonia gas (250 g) was introduced at room temperature over a period of 2.5 hours. The solution generated heat and the reaction proceeded slowly. Thereafter, the reaction mixture was stirred at room temperature for 5.5 hours, and was further left standing for 3 days. After the reaction was completed, the ethanol was distilled off to obtain 213 g of a yellow liquid (crude yield: 89%). Its structure was identified by ¹H-NMR in IR to ascertain that the desired diamino compound was obtained.

(b) Synthesis of Maleinamic Acid:

Two types of solutions obtained by dissolving respectively 104 g (0.5 mol) of the diamino compound obtained above and 98 g (1 mol) of maleic anhydride in 800 ml of dimethylformamide were dropwise added in equal portions to 400 ml of ice-cooled dimethylformamide over a period of 8 hours with stirring under ice cooling. After the addition was completed, the mixture was further stirred for 2 hours. Thereafter, the mixture was left standing overnight, and then concentration of dimethylformamide and filtration were carried out to obtain 180 g of a compound (crude yield: 89%). Its structure was identified by ¹H-NMR in IR to ascertain that the desired maleinamic acid was obtained.

(c) Synthesis of Exemplified Compound 30:

101 g (0.25 mol) of the maleinamic acid obtained above, 255 g (2.5 mols) of acetic anhydride and 12.5 g of sodium acetate were mixed, and these were stirred at 60° C. for 5 hours. After the reaction was completed, most of the acetic acid and acetic anhydride were distilled off under reduced pressure, and the resulting residue was extracted with chloroform. Thereafter, the liquid extract obtained was concentrated, and the precipitate formed was filtered off to obtain 55 g of a compound (crude yield: 60%). Its structure was identified by ¹H-NMR in IR to ascertain that the desired Exemplified Compound 30 was obtained.

### Synthesis Example 2

### Synthesis of Exemplified Compounds 31 and 32

The ethylene glycol diglycidyl ether used in Synthesis Example 1 was changed to trimethylolpropane polyglycidyl ether (EX-321, available from Nagase ChemteX Corporation. Except for this, synthesis was carried out in entirely the same manner as in Synthesis Example 1 through the courses (a), (b) and (c) to obtain a compound.

This compound was analyzed by high-speed liquid chromatography and ascertained to be a mixture of some components. Then, analysis with a liquid chromatograph/mass analyzer showed that a mixture of the following Exemplified Compound 31 and Exemplified Compound 32 was obtained.

### Exemplified Compound 31

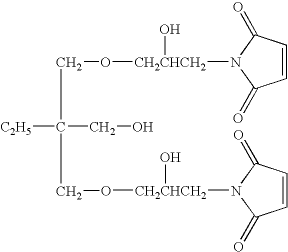

In the Exemplified Compound 31, Z of the general formula (I) corresponds to a trimethylolpropane residue (shown below).

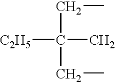

### Exemplified Compound 32

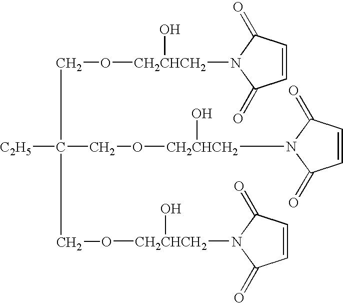

In the Exemplified Compound 32, Z of the general formula (I) corresponds to a trimethylolpropane residue (shown below).

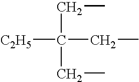

### Synthesis Example 3

### Synthesis of Exemplified Compound 33

The maleic anhydride used in Synthesis Example 1 was changed to itaconic anhydride. Except for this, synthesis was carried out in entirely the same manner as in Synthesis Example 1 through the courses (a), (b) and (c) to obtain a compound. This compound was analyzed by high-speed liquid chromatography and with a liquid chromatograph/mass analyzer to ascertain that the following Exemplified Compound 33 was obtained.

### Exemplified Compound 33

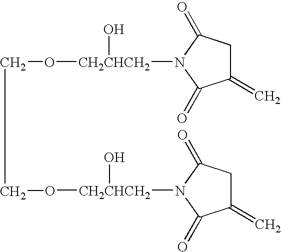

In the Exemplified Compound 33, Z of the general formula (I) corresponds to an ethylene glycol residue (shown below).

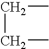

### Synthesis Example 4

### Synthesis of Exemplified Compounds 34 and 35

The maleic anhydride and trimethylolpropane polyglycidyl ether used in Synthesis Example 2 were changed for itaconic anhydride and glycerol polyglycidyl ether (EX-313, available from Nagase ChemteX Corporation), respectively. Except for this, synthesis was carried out in entirely the same manner as in Synthesis Example 2 through the courses (a), (b) and (c) to obtain a compound. This compound was analyzed by high-speed liquid chromatography and with a liquid chromatograph/mass analyzer to ascertain that a mixture of the following Exemplified Compound 34 and Exemplified Compound 35 was obtained.

### Exemplified Compound 34

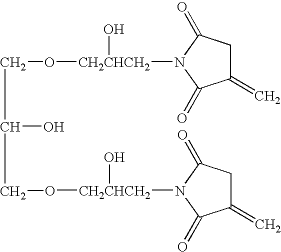

In the Exemplified Compound 34, Z of the general formula (I) corresponds to a glycerol residue (shown below).

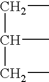

### Exemplified Compound 35

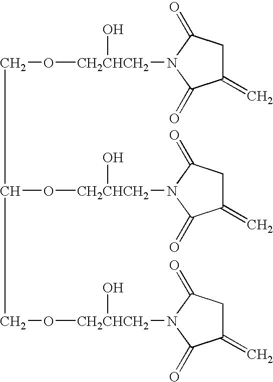

In the Exemplified Compound 35, Z of the general formula (I) corresponds to a glycerol residue (shown below).

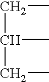

### Synthesis Example 5

### Synthesis of Exemplified Compound 36

As a diamino compound, 150 g (0.25 mol) of a polyoxyalkylenediamino compound (JEFFARMINE ED 600, available from Huntsman International LLC.). Except for this, synthesis was carried out in entirely the same manner as in Synthesis Example 1, the course (b), to obtain 201 g of bismaleamic acid derivative.

177 g (0.22 mol) of the bismaleamic acid derivative obtained above was used. Except for this, synthesis was carried out in entirely the same manner as in Synthesis Example 1, the course (c). Thereafter, alumina chromatograph purification and sellaite treatment were carried out to obtain 50 g of a liquid. Its structure was identified by gel permeation chromatography (GPC) and H-NMR in IR to ascertain that the following Exemplified Compound 36 was obtained. The results of identification made by the ¹H-NMR were as follows: A peak (A) around 1 ppm due to —CH₃—, a peak (B) around 3 to 4 ppm due to —CH— and —CH₂— and a peak (C) around 6 to 7 ppm due HC═CH— were in an integral intensity ratio A:B:C of 13.8:48:4.

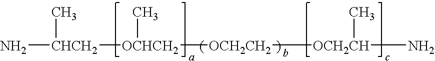

Average number of propylene oxide chains: a+c+1=about 4.6.
Average number of ethylene oxide chains: b=about 9.

### Exemplified Compound 36

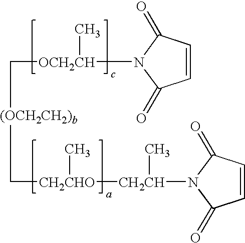

In the Exemplified Compound 36, Z of the general formula (I) corresponds to a polyethylene glycol residue having an average molecular weight of about 400, represented by —(O—CH₂—CH₂)_{b}—. The average unit number b is about 9. R₁ and R₂ in A and B of the general formula (I) are each a methyl group, and the value of a+c is about 3.6.

### Synthesis Example 6

### Synthesis of Exemplified Compound 38

Maleic anhydride used in Synthesis Example 5 was changed to itaconic anhydride. Except for this, synthesis was carried out in entirely the same manner as in Synthesis Example 5 to obtain a liquid. Its structure was identified by gel permeation chromatography (GPC) and ¹H-NMR in IR to ascertain that the following Exemplified Compound 38 was obtained.

### Exemplified Compound 38

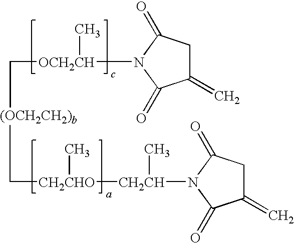

In the Exemplified Compound 38, Z of the general formula (I) corresponds to a polyethylene glycol residue having an average molecular weight of about 400, represented by —(O—CH₂—CH₂)_{b}—. The average unit number b is about 9. R₁ and R₂ in A and B of the general formula (I) are each a methyl group, and the value of a+c is about 3.6.

### Examples 1 to 20 &

### Comparative Examples 1 to 8

Such components as shown in Tables 1 and 2 were mixed and thoroughly stirred, followed by pressure filtration carried out using a filter of 1.2 microns in pore size to prepare liquid compositions of Examples 1 to 20 and Comparative Examples 1 to 8. As polymerizable substances used in Comparative Examples 1 to 8, Comparative Compounds 1 and 2 shown below were used. In the present invention, the recording by which each pixel of images formed at 600×600 dpi is completely filled up with about 5 pl dots is called 100% solid.

### Comparative Compound 1

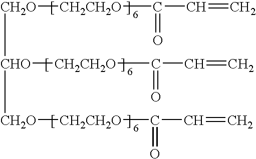

### Comparative Compound 2

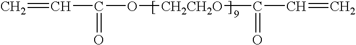

TABLE 1
Compositions of Examples 1 to 10 and Comparative Examples 1 to 4 (unit: part(s) by mass)

| | | | | | | | | | | | Comparative | | | |
|---|---|---|---|---|---|---|---|---|---|---|---|---|---|---|
| | Example | | | | | | | | | | Example | | | |
| | 1 | 2 | 3 | 4 | 5 | 6 | 7 | 8 | 9 | 10 | 1 | 2 | 3 | 4 |
| Polymerizable substance: | | | | | | | | | | | | | | |
| Exemplified Compound 30 | 40 | — | — | — | — | 40 | — | — | — | — | — | — | — | — |
| Exemplified Compounds 31, 32 | — | 40 | — | — | — | — | 40 | — | — | — | — | — | — | — |
| Exemplified Compounds 34, 35 | — | — | 40 | — | — | — | — | 40 | — | — | — | — | — | — |
| Exemplified Compound 36 | — | — | — | 40 | — | — | — | — | 40 | — | — | — | — | — |
| Exemplified Compound 38 | — | — | — | — | 40 | — | — | — | — | 40 | — | — | — | — |
| Comparative Compound 1 | — | — | — | — | — | — | — | — | — | — | 40 | — | 40 | — |
| Comparative Compound 2 | — | — | — | — | — | — | — | — | — | — | — | 40 | — | 40 |
| Polymerization initiator: | | | | | | | | | | | | | | |
| Exemplified Compound 25 | 8 | 8 | 8 | 8 | 8 | — | — | — | — | — | 8 | 8 | — | — |
| Exemplified Compound 26 | — | — | — | — | — | 8 | 8 | 8 | 8 | 8 | — | — | 8 | 8 |
| Diluent: | | | | | | | | | | | | | | |
| ACMO | 52 | 52 | 52 | 52 | 52 | 52 | 52 | 52 | 52 | 52 | 52 | 52 | 52 | 52 |
| Water: | | | | | | | | | | | | | | |
| Ion-exchange water | 0 | 0 | 0 | 0 | 0 | 0 | 0 | 0 | 0 | 0 | 0 | 0 | 0 | 0 |

TABLE 2
Compositions of Examples 11 to 20 and Comparative Examples 5 to 8 (unit: parts by mass)

| | | | | | | | | | | | Comparative | | | |
|---|---|---|---|---|---|---|---|---|---|---|---|---|---|---|
| | Example | | | | | | | | | | Example | | | |
| | 11 | 12 | 13 | 14 | 15 | 16 | 17 | 18 | 19 | 20 | 5 | 6 | 7 | 8 |
| Polymerizable substance: | | | | | | | | | | | | | | |
| Exemplified Compound 30 | 30 | — | — | — | — | 30 | — | — | — | — | — | — | — | — |
| Exemplified Compounds 31, 32 | — | 30 | — | — | — | — | 30 | — | — | — | — | — | — | — |
| Exemplified Compounds 34, 35 | — | — | 30 | — | — | — | — | 30 | — | — | — | — | — | — |
| Exemplified Compound 36 | — | — | — | 30 | — | — | — | — | 30 | — | — | — | — | — |
| Exemplified Compound 38 | — | — | — | — | 30 | — | — | — | — | 30 | — | — | — | — |
| Comparative Compound 1 | — | — | — | — | — | — | — | — | — | — | 30 | — | 30 | — |
| Comparative Compound 2 | — | — | — | — | — | — | — | — | — | — | — | 30 | — | 30 |
| Polymerization initiator: | | | | | | | | | | | | | | |
| Exemplified Compound 25 | 4 | 4 | 4 | 4 | 4 | — | — | — | — | — | 4 | 4 | — | — |
| Exemplified Compound 26 | — | — | — | — | — | 4 | 4 | 4 | 4 | 4 | — | — | 4 | 4 |
| Diluent: | | | | | | | | | | | | | | |
| ACMO | 10 | 10 | 10 | 10 | 10 | 10 | 10 | 10 | 10 | 10 | 10 | 10 | 10 | 10 |
| Water: | | | | | | | | | | | | | | |
| Ion-exchange water | 56 | 56 | 56 | 56 | 56 | 56 | 56 | 56 | 56 | 56 | 56 | 56 | 56 | 56 |

In Tables 1 and 2:

ACMO stands for acryloyl morpholine;

Exemplified Compounds 31 and 32 were used as a mixture of 1:1 in mass ratio; and

Exemplified Compounds 34 and 35 were used as a mixture of 1:1 in mass ratio.

—Evaluation of Film Forming Properties of Liquid Composition—

Using the liquid compositions shown in Tables 1 and 2, their film forming properties were evaluated in the following way. To commercially available PET (polyethylene terephthalate) films, the liquid compositions of Examples 1 to 20 and Comparative Examples 1 to 8 were applied using a bar coater to be 20 g/m² each in coverage. The PET films thus obtained were irradiated with ultraviolet rays by using a UV irradiator. The UV lamp used was a UV curability evaluation device Model LH6B (manufactured by FUSION UV Systems Inc.), and its intensity at the irradiation position was 1,500 mW/cm². The PET films were transported at a speed of 0.2 m/second. The pencil hardness of the films thus formed was measured with a commercially available pencil hardness tester (HEIDON-14D, manufactured by Shinto Kagaku). The measurement results are shown in Tables 3 and 4. The pencil hardness test accords with JIS.

TABLE 3
Coating Films of Examples 1 to 10,
Measurement Results of Pencil Hardness
Evaluation item: Pencil hardness
Test method: According to JIS
Example

| 1 | 2 | 3 | 4 | 5 | 6 | 7 | 8 | 9 | 10 |
|---|---|---|---|---|---|---|---|---|---|
| 3H | 3H | 3H | 3H | 3H | 3H | 3H | 3H | 3H | 3H |

TABLE 4
Coating Films of Examples 11 to 20,
Measurement Results of Pencil Hardness
Evaluation item: Pencil hardness
Test method: According to JIS
Example

| 11 | 12 | 13 | 14 | 15 | 16 | 17 | 18 | 19 | 20 |
|---|---|---|---|---|---|---|---|---|---|
| 2H | 2H | 2H | 2H | 2H | 2H | 2H | 2H | 2H | 2H |

As shown in the results of the examples in Tables 3 and 4, pencil hardness of each film formed as above having no problem in practical use was achieved without regard to non-water base (examples 1 to 10) or water base (examples 11 to 20). The films formed using the liquid compositions of Comparative Examples 1 to 8 were not completely be fixed to the PET films, and their pencil hardnesses were not measurable with the pencil hardness tester.

### Examples 21 to 32 &

### Comparative Examples 9 and 10

Cyan pigment dispersions were prepared in the following way. C.I. Pigment Blue 15:3 was used as a pigment, and a styrene-acrylic acid-ethyl acrylate random polymer (average molecular weight: 3,500; acid value: 150) was used as a dispersant. These were put to dispersion by means of a bead mill to obtain a cyan pigment dispersion having a pigment solid content of 10% by mass and a P/B ratio of 3/1, a proportion of the pigment to the binder. The pigment had an average particle diameter of 120 nm as measured with a laser beam scattering type particle diameter measuring instrument (ELS-8000, manufactured by Otsuka Electronics Co., Ltd.).

Next, such components as shown in Table 5 were mixed and thoroughly stirred, followed by pressure filtration carried out using a filter of 0.50 μm in pore size to prepare inks to be used in Examples 21 to 32 and Comparative Examples 9 and 10. The pH of each ink was so adjusted as to be finally 8.5, by using an aqueous 0.2-normal sodium hydroxide solution. As polymerizable substances used in Comparative Examples 9 and 10, Comparative Compounds 1 and 2 shown below were used.

TABLE 5
Compositions of Examples 21 to 32 and Comparative Examples 9 and 10 (unit: parts by mass)

| | | | | | | | | | | | | | Comparative | |
|---|---|---|---|---|---|---|---|---|---|---|---|---|---|---|
| | Example | | | | | | | | | | | | Example | |
| | 21 | 22 | 23 | 24 | 25 | 26 | 27 | 28 | 29 | 30 | 31 | 32 | 9 | 10 |
| Coloring material: | | | | | | | | | | | | | | |
| Pigment dispersion | 40 | 40 | 40 | 40 | 40 | 40 | 40 | 40 | 40 | 40 | 40 | 40 | 40 | 40 |
| Polymerizable substance: | | | | | | | | | | | | | | |
| Exemplified Compound 30 | 15 | — | — | — | — | — | 25 | — | — | — | — | — | — | — |
| Exemplified Compounds 21, 22 | — | 15 | — | — | — | — | — | 25 | — | — | — | — | — | — |
| Exemplified Compound 33 | — | — | 15 | — | — | — | — | — | 25 | — | — | — | — | — |
| Exemplified Compounds 23, 24 | — | — | — | 15 | — | — | — | — | — | 25 | — | — | — | — |
| Exemplified Compound 36 | — | — | — | — | 15 | — | — | — | — | — | 25 | — | — | — |
| Exemplified Compound 38 | — | — | — | — | — | 15 | — | — | — | — | — | 25 | — | — |
| Comparative Compound 1 | — | — | — | — | — | — | — | — | — | — | — | — | 15 | — |
| Comparative Compound 2 | — | — | — | — | — | — | — | — | — | — | — | — | — | 15 |
| Polymerization initiator: | | | | | | | | | | | | | | |
| Exemplified Compound 25 | — | — | — | — | — | — | 2 | 2 | 2 | 2 | 2 | 2 | — | — |
| Exemplified Compound 26 | 3 | 3 | 3 | 3 | 3 | 3 | — | — | — | — | — | — | 3 | 3 |
| Diluent: | | | | | | | | | | | | | | |
| HEAA | 10 | 10 | 10 | 10 | 10 | 10 | — | — | — | — | — | — | 10 | 10 |
| Organic solvent: | | | | | | | | | | | | | | |
| EG | — | — | — | — | — | — | 10 | 10 | 10 | 10 | 10 | 10 | — | — |
| Water: | | | | | | | | | | | | | | |
| Ion-exchange water | 32 | 32 | 32 | 32 | 32 | 32 | 23 | 23 | 23 | 23 | 23 | 23 | 32 | 32 |

In Table 5:

The pigment solid content in each ink was so adjusted as to be in an amount of 4% by mass;

Exemplified Compounds 21 and 22 were used as a mixture of 1:1 in mass ratio;

Exemplified Compounds 23 and 24 were used as a mixture of 1:1 in mass ratio;

HEAA stands for hydroxyethyl acrylamide; and

EG stands for ethylene glycol.

### Comparative Compound 1

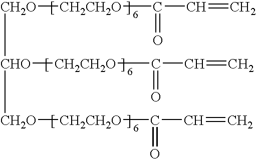

### Comparative Compound 2

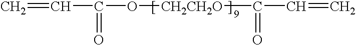

The inks prepared as described above were evaluated in the following way.

Ink Jet Recording Apparatus for Evaluation

An on-demand type ink jet recording apparatus PIXUS 550i (manufactured by CANON INC.), in which thermal energy corresponding to recording signals is applied to ink to eject it, was so modified as to have such a constitution as shown in FIG. 1. Specifically, UV lamps were mounted which adjoin the recording head and excite mercury lamps without electrodes from the outside by using microwaves. Using this ink jet recording apparatus, evaluation was made by evaluation methods, and according to evaluation criteria, as shown in the following (1) to (3). D valves were used as the UV lamps. Their intensity at the irradiation position was 1,500 mW/cm².

(1) Ink Curing Performance

(1)-1: Fixing Performance

Using the respective cyan inks for Examples 21 to 32 and Comparative Examples 9 and 10 and the above ink jet recording apparatus, 100%-solid images were formed on offset recording paper OK Kinfuji (available from Mitsubishi Paper Mills Limited). This recording medium with the images was irradiated with ultraviolet rays by using a UV irradiator, under the same conditions as in the case where the liquid composition was applied. After 10 seconds have passes from the completion of recording, Silbon paper was placed on the recording medium with the images and a load of 40 g/cm² was applied on the recorded surface, and in this state, the Silbon paper was pulled. It was visually inspected whether or not any stains occurred on the non-recorded areas (white background area) of the recording medium and the Silbon paper as a result of the scratching of recorded areas, thus evaluation was made. Evaluation criteria of fixing performance are as shown below. The evaluation results are shown in Table 6.

A: Any stained area due to the scratching was not seen.
B: Stained areas due to the scratching were hardly seen.
C: Stained areas due to the scratching were conspicuous.

(1)-2, Marker Resistance

Using the respective cyan inks for Examples 21 to 32 and Comparative Examples 9 and 10 and the above ink jet recording apparatus, 12-point characters were recorded on, PPC paper (available from CANON INC.). After 1 minute has passed after the completion of recording, character areas were marked once with a highlighter marker SPOT WRITER YELLOW (available from PILOT Corporation) at usual writing pressure. It was visually inspected whether or not any disorder of characters was seen, thus evaluation was made. Evaluation criteria of marker resistance are as shown below. The evaluation results are shown in Table 6.

A: no disturbance of characters due to the marker occurred.
B: Disturbance of characters slightly occurred.
C: Disturbance of characters seriously occurred.

(2) Ejection Stability

Using the respective cyan inks for Examples 21 to 32 and Comparative Examples 9 and 10 and the above ink jet recording apparatus, horizontal lines were continuously recorded on PPC paper (available from CANON INC.). Thereafter, line thickness and ink-droplet impact position (dot position) were 0.5 visually inspected, thus evaluation was made.

Evaluation criteria of ejection stability are as shown below. The evaluation results are shown in Table 6.

A: No change in line thickness was seen, and no dot miss-alignment was also seen at all.
B: Thick lines were somewhat seen, but at the level that no problem occurred in practical use.
C: Thin lines were seen, and dot miss-alignment as well was somewhat seen.

(3) Storage Stability

The respective cyan inks for Examples 21 to 32 and Comparative Examples 9 and 10 were put into TEFLON (registered trademark) containers, which were then hermetically sealed. These were stored in a 60° C. oven for a month in a dark place. Average particle diameters of pigments before and after storage were compared, thus evaluation was made. Evaluation criteria of storage stability are as shown below. The evaluation results are shown in Table 6.

A: Change in average particle diameter was within ±10% before and after storage.
B: Change in average particle diameter was more than ±10% and within ±15% before and after storage.
C: Change in average-particle diameter was more than 15% before and after storage.

TABLE 6
Evaluation Results

| | | | | | | | | | | | | | Comparative | |
|---|---|---|---|---|---|---|---|---|---|---|---|---|---|---|
| | Example | | | | | | | | | | | | Example | |
| | 21 | 22 | 23 | 24 | 25 | 26 | 27 | 28 | 29 | 30 | 31 | 32 | 1 | 2 |
| Ink curing performance: | | | | | | | | | | | | | | |
| Fixing performance | A | A | A | A | A | A | A | A | A | A | A | A | A | B |
| Marker resistance | B | A | A | A | A | A | B | A | A | A | A | A | B | B |
| Ejection stability: | A | A | A | A | A | A | A | A | A | A | A | A | C | C |
| Storage stability: | A | A | A | A | A | A | A | A | A | A | A | A | C | C |

### Example 33

A yellow pigment dispersion and a magenta pigment dispersion were prepared in the same manner as the preparation of the cyan pigment dispersion used in Example 21.

Preparation of Yellow Pigment Dispersion

A yellow pigment dispersion having a pigment solid content of 10% by mass, a P/B ratio of 3/1 and an average particle diameter of 130 nm was prepared in entirely the same manner as in the preparation of the cyan pigment dispersion except that C.I. Pigment Yellow 13 was used as the pigment.

Preparation of Magenta Pigment Dispersion

A magenta pigment dispersion having a pigment solid content of 10% by mass, a P/B ratio of 3/1 and an average particle diameter of 125 nm was prepared in entirely the same manner as in the preparation of the cyan pigment dispersion except that C.I. Pigment Red 122 was used as the pigment.

Next, a yellow ink for Example 33 was prepared in entirely the same manner as in Example 21 except that the cyan pigment dispersion used therein was changed to the yellow pigment dispersion obtained above. A magenta ink for Example 33 was also prepared in entirely the same manner as in Example 21 except that the cyan pigment dispersion used therein was changed for the magenta pigment dispersion obtained as above.

The cyan ink for Example 21 was combined with the yellow ink and magenta ink obtained above to make up an ink set for Example 33. Using this ink set and the same ink jet recording apparatus as used in Example 21, images were recorded on offset recording paper OK Kinfuji (available from Mitsubishi Paper Mills Limited). Specifically, 100%-solid images of yellow and magenta, and secondary-color red images formed form yellow 100%-solid images and magenta 100%-solid images were recorded. In respect of yellow, magenta and red portions of the images thus formed, fixing performance was evaluated by the same evaluation methods, and according to the same evaluation criteria, as in Example 21 (which were designated as Examples 33Y, 33M and 33R, respectively). In respect of the yellow ink and magenta ink, the ejection stability and storage stability also were evaluated by the same evaluation methods, and according to the same evaluation criteria, as in Example 21. The evaluation results are shown in Table 7.

TABLE 7

| | Example | | |
|---|---|---|---|
| | 33Y | 33M | 33R |
| Fixing performance: | A | A | A |
| Ejection stability: | A | A | — |
| Storage stability: | A | A | — |

As described above, according to the present invention, inks and liquid compositions can be provided which have a good curability by the active-energy, can achieve practical curing performance even when prepared as inks containing coloring materials, have superior fixing performance and marker resistance, and are superior in ejection stability and storage stability. The above Examples have been given in order to describe the basic constitution of the present invention. Needless to say, inks having the same performance as those in the above Examples can be provided even when, e.g., the dye is used as the coloring material.

This application claims priority from Japanese Patent Application No. 2005-287799 filed on Sep. 30, 2005, which is hereby incorporated by reference herein.

1. An active-energy radiation-polymerizable substance represented by the following general formula (I): 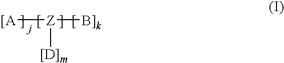
wherein Z is a residue of a dihydric to hexahydric polyol, j is 1 to 6, k is 0 to 2, and m is 0 to 2;
A is a group represented by the following general formula (II): 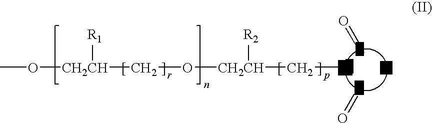
wherein n is 0 to 5; p is 0 to 1; R₁ and R₂ are each independently a hydrogen atom, a methyl, group or a hydroxyl group; r is 0 to 1; and X is a divalent group constituted of 2 to 5 carbon atoms in which at least one of the carbon atoms adjoining to the carbonyl carbon has a carbon-carbon double bond;
B is a group represented by the following general formula (III): 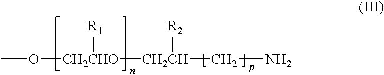
wherein n is 0 to 5; p is 0 to 1; and R₁ and R₂ are each independently a hydrogen atom, a methyl group or a hydroxyl group; and
D is a group represented by the following general formula (IV): 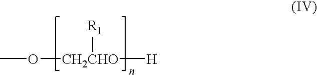
wherein n is 0 to 5; and R₁ is a hydrogen atom, a methyl group or a hydroxyl group.

2. The active-energy radiation-polymerizable substance according to claim 1, wherein, in the general formula (II), —X— is a group represented by the following chemical formula (1) or chemical formula (2):

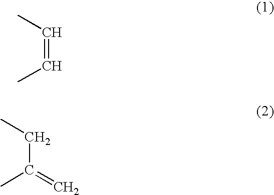

3. The active-energy radiation-polymerizable substance according to claim 1, wherein the active-energy radiation-polymerizable substance has both an ethylene oxide group and a propylene oxide group.

4. The active-energy radiation-polymerizable substance according to claim 1, wherein, in the general formula (I), j is 3 to 6.

5. An active-energy radiation-curable liquid composition comprising at least an active-energy radiation-polymerizable substance;
wherein the active-energy radiation-polymerizable substance is the active-energy radiation-polymerizable substance according to claim 1.

6. The active-energy radiation-curable liquid composition according to claim 5, further comprising a polymerization initiator capable of generating a radical by irradiation with an active-energy radiation.

7. The active-energy radiation-curable liquid composition according to claim 5, further comprising water.

8. An active-energy radiation-curable ink comprising at least an active-energy radiation-polymerizable substance and a coloring material;
wherein the active-energy radiation-polymerizable substance is the active-energy radiation-polymerizable substance according to claim 1.

9. The active-energy radiation-curable ink according to claim 8, further comprising a polymerization initiator capable of generating a radical by irradiation with an active-energy radiation.

10. The active-energy radiation-curable liquid composition according to claim 8, further comprising water.

11. The active-energy radiation-curable liquid composition according to claim 8, which is used for ink jet recording.

12. An ink jet recording method having the step of ejecting an ink to apply the ink to a recording medium and the step of irradiating the recording medium to which the ink has been applied, with an active-energy radiation to cure the ink,
wherein the ink is the active-energy radiation-curable ink according to claim 11.

13. The ink jet recording method according to claim 12, wherein the ink is ejected by action of thermal energy to be applied to the recording medium.

14. An ink cartridge, comprising an ink storage portion for storing ink therein, wherein the ink comprises the active-energy radiation-curable ink according to claim 11.

15. A recording unit, comprising an ink storage portion for storing ink therein and a recording head for ejecting the ink, wherein the ink comprises the active-energy radiation-curable ink according to claim 11.

16. An ink jet recording apparatus, comprising a means for applying the active-energy radiation-curable ink to a recording medium and a means for irradiating the recording medium to which the ink has been applied with an active-energy radiation to cure the ink, wherein the ink comprises the active-energy radiation-curable ink according to claim 11.
